(19) United States
(12) Patent Application Publication
(10) Pub. No.: US 2024/0325757 A1
(43) Pub. Date: Oct. 3, 2024

(54) DEVICES AND METHODS FOR IMPROVING HEADACHE DISORDERS

(71) Applicant: Mudjala Medical Pty Ltd, Bronte (AU)

(72) Inventors: David J. ROBINSON, Bronte (AU); Tracy Cameron, Toronto (CA); Emest Mantes, The Colony, TX (US)

(73) Assignee: Mudjala Medical Pty Ltd, Bronte (AU)

(21) Appl. No.: 18/499,927

(22) Filed: Nov. 1, 2023

Related U.S. Application Data
(63) Continuation of application No. 17/065,269, filed on Oct. 7, 2020.
Application No. 17/065,269 is a continuation of application No. PCT/US2019/027245, filed on Apr. 12, 2019.

(30) Foreign Application Priority Data
Apr. 12, 2018 (AU) 2018901210

(51) Int. Cl. A61N 1/36; A61B 17/34; A61N 1/05

(52) U.S. Cl. CPC A61N 1/36075; A61B 17/3468; A61N 1/0529; A61N 1/36139

(57) ABSTRACT

Methods and devices for fabricating, implanting and explanting stimulators for neuromodulation and for applying them for peripheral nerve stimulation applications, including improving headache disorders in a patient suffering therefrom, by electrically modulating neural tissue in a minimally invasive fashion are provided.

### CROSS-REFERENCE TO RELATED APPLICATIONS

This application is a continuation of U.S. patent application Ser. No. 17/065,269, filed Oct. 7, 2020, which is a continuation of PCT Application No. PCT/US2019/027245, filed Apr. 12. 2019, which claims the benefit of AU Provisional Application No. 20/189,01210, filed Apr. 12, 2018, the entirety of which applications are incorporated herein by reference.

### BACKGROUND OF THE INVENTION

### 1. Field of the Invention

The present disclosure relates to electrical stimulators for neuromodulation, delivery systems for implanting electrical stimulators, and methods of alleviating headaches.

The International Neuromodulation Society defines therapeutic neuromodulation as “the alteration of nerve activity through targeted delivery of a stimulus, such as electrical stimulation or chemical agents, to specific neurological sites in the body.” The use of electrical stimulation to modulate neural activity, sometimes called “neurostimulation” is practiced for a range of therapies, some related to the treatment of chronic pain, while others address issues such as incontinence, overactive bladder, epilepsy, movement and other brain-based disorders and organ control. The practice of neurostimulation can be broadly divided into therapies which target the central nervous system e.g. Spinal Cord Stimulation (SCS) or Deep Brain Stimulation (DBS), and those which target the Peripheral Nervous System, broadly known as Peripheral Nerve Stimulation or PNS therapies.

Chronic migraine one of several headache disorders, identified by disabling migraine symptoms being present for four or more hours per day on 15 or more days per month for three or more months (IHS Classification), ranks in the top 20 of the world's most disabling illnesses (Migraine Research Foundation). More than 90% of migraine patients (called migraineurs) are unable to work or function normally during a migraine attack.

There are currently no universally effective treatments for migraineurs. The typical therapy path for migraineurs begins with patients beginning to recognize the symptoms of migraine, they often identify and avoid the “triggers” that seem to spark the onset of an attack. When trigger avoidance is impossible or becomes ineffective, patients will often turn to non-specific migraine medications, including over-the-counter and/or prescription pain medicines such as non-steroidal anti-inflammatoires, or acetaminophen. When these become ineffective they often seek professional advice.

There are two classes of approved migraine-specific acute drugs (dosed during an attack to treat migraine), Triptans and Ergots, as well as certain combination drugs. Once an appropriate, patient-specific drug and dosage pattern is found (This trial-and-error process can sometimes take several months or even years); many patients find that these drugs are effective at stopping periodic migraine attacks. However, they come with some common side effects including nausea, jaw, neck or chest tightness, rapid heart rate, fatigue and paraesthesia. Further, triptans are contraindicated for patients with certain cardiovascular disorders. Additionally, there are five prophylactic drugs (dosed regularly to prevent migraine attacks). These include topiramate, propranolol, timolol, divalproex sodium, and onabotulinum toxin A.

Other classes of drugs that have been used to treat and/or prevent migraines include Beta-blockers, calcium channel antagonists, antidepressants, anticonvulsants, serotonergic agents; many of these drugs are used off-label, meaning they are not specifically approved for—but have, in certain cases, been effective in—the treatment or prevention of migraine.

Getting the patient-specific drugs or drug combinations and dosages correct can take years. An estimated 3-5 million patients in the US are currently taking prophylactic migraine drugs. Finally, when no drugs seem to be effective, or the side-effects are deemed intolerable, the most severe and intractable headache disorders patients (who haven't yet given up seeking therapy) may be treated with repeated chemical nerve blocks (injections), or be referred to surgeons who can attempt to treat the disease with various forms of destructive nerve procedures or invasive neuromodulation (spinal cord, peripheral, or deep-brain stimulation-all currently performed “off-label” and subject to serious surgical complications).

Although clinical data has shown that Occipital Nerve Stimulation (ONS) improves the pain and disability caused by headache disorders, current technology limitations have not allowed this therapy to become a standard treatment. In a recent study of ONS for headache disorders patients who had failed multiple prophylactic drugs, headache days were reduced by 6.1 days (vs. 3.0 for placebo). However, adverse events were high (68% of all patients) and included lead migration (18.7%), erosion (5.6%), infection (6.5%), and persistent implant site pain (21.5%).

### 2. Background Art

The subject matter of this application relates to that of commonly owned Patent Cooperation Treaty application PCT/US16/27570, the entirety of which application is incorporated by reference. See also, U.S. Pat. Nos. 8,936,630; 5,215,086; 9,002,458; and US 2016/0339239.

### SUMMARY OF THE INVENTION

The present disclosure generally relates to methods and devices for implanting Peripheral Nerve Stimulation (PNS) devices in general, and in particular the implanting of an electrical ribbon stimulator (also referred to herein as a “ribbon stimulator”) or a ribbon-style stimulation lead attached to a conventional Implantable Pulse Generator (IPG) in a minimally invasive fashion. In general, the term ribbon stimulator used herein can refer to either the ribbon lead or the ribbon stimulator. Such devices may be implanted for improving headache disorders, chronic pain of peripheral origin or other disorder in a patient suffering therefrom by electrically modulating neural tissue

Embodiments of this invention for the minimally invasive, subcutaneous placement of a neurostimulation device described herein generally In one embodiment of this invention, a method of delivering PNS for therapeutic benefit comprises making a superficial incision in the skin in the region of the target nerve or group of nerve fibers, for the therapy in view, and inserting a ribbon stimulator through said incision such that it can stimulate the target nerve or group of nerve fibers.

In certain embodiments the implant site will be in the layer of adipose tissue (hypodermis) below the skin (dermis) and above the fascia. In one embodiment, a method of improving headache disorders in a patient suffering therefrom comprises inserting an all-on-head ribbon stimulator through skin on the back of a patient's head and advancing the ribbon stimulator under the skin at or above the occipital ridge. The method further includes positioning the ribbon stimulator at a target implant site adjacent to a target nerve associated with headache disorders, In another embodiment, a method of treating Chronic Back Pain (CBP) in a patient suffering therefrom comprises inserting one or more ribbon stimulators through the skin in the region when the patient feels the CBP such that all the nerve fibers in that area are able to be stimulated by the one or more ribbon stimulators

In certain embodiments, the target implant site is in the adipose tissue above the galia (fascia) and below the dermis. The method further comprises self-anchoring the ribbon stimulator to the target implant site. Once the ribbon stimulator has been inserted to the target implant site, the method comprises delivering a therapy electrical signal to the target nerve to improve headache disorders.

Headache disorders can refer to but are not limited to migraine, chronic migraine, episodic migraine, cluster headache, chronic cluster headache, episodic cluster headache Chronic Pain disorders refer to, but are not limited to, back pain, pain of trunk and limbs, thoracotomy pain, fibromyalgia, shingles, nerve damage and arthritis.

In a first aspect of the present invention, a method for implanting a ribbon stimulator over a patient's occipital or other target nerves comprises identifying a target location on a the patient's head, typically on a back of the patient's head when the target nerves are the occipital nerves. A superficial incision is formed at the target location, and the ribbon stimulator is advanced through the superficial incision beneath connective tissue of the patient's scalp to position electrodes on the ribbon stimulator over the occipital nerves. The method will typically further comprise at least one additional step, often comprising two or more of the additional steps which will now be described.

A first additional step comprises pushing the ribbon stimulator or an associated advancement tool so that a blunt leading or distal end of the stimulator or the advancement tool dissects adjacent tissue layers of the scalp as the blunt leading edge is advanced.

A second additional step comprises pre-forming a pocket from the target location to a location over the occipital nerves, where the ribbon stimulator is then typically advanced through the pocket from the target location to the occipital nerves.

A third additional method step comprises cutting the ribbon stimulator to a pre-selected length prior to advancing the ribbon stimulator. In this way, the need to maintain an inventory of ribbon stimulators having different lengths suitable for different patients can be reduced or eliminated.

A fourth additional method step comprises injecting an anesthetic agent at a location remote from the target location of the incision, typically at or near the occipital nerves as described below.

A fifth additional method step comprises tracking the location of the ribbon stimulator as the ribbon stimulator is advanced from the incision to the occipital nerves.

In specific instances, the ribbon stimulator implanted by the methods herein consists of an integrated structure comprising an elongate ribbon body, one or more electrodes formed on the elongate ribbon body, and electrical circuitry on the ribbon body coupled to the one or more electrodes.

In still further specific instances, the target location is above the fascia, epicranial aponeurosis or galea and below the dermis.

In still other specific embodiments of the methods of the present invention, advancing the ribbon stimulator comprises at least pushing the ribbon stimulator so that a blunt leading end of the stimulator dissects adjacent tissue layers of the scalp as it is advanced. For example, the ribbon stimulator may be removably attached to an advancement tool, and the advancement tool used to push the leading edge of the ribbon stimulator through the tissue layers. After reaching the occipital nerves, the ribbon stimulator may be detached from the advancement tool.

Often, the ribbon stimulator will be temporarily stiffened while it is being advanced by the advancement tool. For example, the ribbon stimulator may be attached along at least a portion of its length to a surface of the advancement tool, such that the advancement tool will stiffen and stabilize the ribbon stimulator as it is being advanced. Temporary attachment of the ribbon stimulator to the advancement tool may be achieved in a variety of ways. For example, the length of the ribbon stimulator may be attached to a length of the advancement tool by a one-way coupling structure which interfaces between the ribbon stimulator and the advancement tool. The one-way coupling structure will allow the advancement tool to push on the ribbon stimulator to advance the ribbon stimulator tissue, while retraction of the advancement tool will detach the ribbon stimulator from the tool. In other embodiments, the ribbon stimulator may be attached to the advancement tool by bonding with a suitable adhesive, preferably one that can be selectively de-bonded when it is desired to detach the ribbon stimulator. In other instances, the ribbon stimulator may be supported and stiffened by a support element such as a support ring or other support element disposed at least around a portion of a periphery of the ribbon stimulator, where the advancement tool may be detachably engaged to the support element to allow advancement of the assembly of the support element and the ribbon stimulator through tissue.

In other embodiments of the implantation methods of the present invention, the method will comprise at least pre-forming a pocket from the target location to a location over the occipital nerves, where the ribbon stimulator may then be advanced through the pocket. The ribbon stimulator may be advanced in a variety of ways. For example, the ribbon stimulator may be pulled through the pocket, where the advancement tools typically has a hook or other grasping element, and the ribbon stimulator has an attachment element such as a loop or other grasping element on a leading edge thereof. In other instances, a second superficial incision may be created at an end of the tissue pocket proximate the target location, where the ribbon stimulator is advanced through the pocket by pulling on a temporary or permanent feature of the ribbon stimulator added either during manufacture or during the conduct of the implantation procedure.

In still further specific embodiments of the methods of implantation herein, the methods will comprise at least cutting the ribbon stimulator to a pre-selected length prior to advancing the ribbon stimulator through tissue. In some instances, selecting the length may be achieved by measuring a distance between the target location of the incision and a location of the occipital nerves. In other instances, the ribbon stimulator may comprise an elongate ribbon body having one or more electrodes positioned along a longitudinal axis thereof, where cutting may comprise cutting laterally across the ribbon body and the electrodes. In such instances, the elongate body will typically have two ends and further comprise electric circuitry at or near one of the two ends. The ribbon body can be then cut at an location between the electric circuitry and the other end of the ribbon body, assuring the continuity between the electric circuitry and the electrodes.

In still further specific embodiments of the implantation methods of the present invention, the methods will comprise at least injecting an anesthetic at a location remote from the target location. Typically, the remote location will be proximate the occipital nerves.

In yet further embodiments of the implantation methods of the present invention, the methods will comprise at least tracking the location of the ribbon stimulator as the ribbon stimulator is advanced. Tracking the location may be achieved in a variety of ways. For example, tracking the location of the ribbon stimulator may comprise monitoring tissue impedance. In particular, tissue impedance may be measured using a pair of electrodes on the ribbon stimulator or on the advancement tool. The measured impedance may be used to determine if the ribbon stimulator or the advancement tool have come into contact with deep fascia or the galea. Impedance generally decreases from the skin in a direction toward the deep fascia. Thus, a significant lowering of the impedance indicates that the electrodes are near or in contact with the fascia, and the physician can be alerted to avoid penetration of the fascia or galea. In other instances, tracking the location of the ribbon stimulator may be accomplished by monitoring nerve activation. Nerve activation may comprise, for example, measuring any one or more of EMG (electromyography), ENG (Electroneurography), TSEP (Somatosensory Evoked Potential), EMAP (Evoked Muscle Action Potential), and ECAP (Evoked Compound Action Potential).

In a second aspect of the present invention, a transcutaneous delivery system comprises a ribbon stimulator and an advancement tool. The ribbon stimulator will typically include an elongate ribbon body and one or more electrodes formed on the elongate ribbon body. The advancement tool will typically comprise an elongate tool body having a flat surface for detachably receiving the elongate ribbon body of the ribbon stimulator, and at least one of the elongate tool body and the ribbon stimulator will typically have a blunt distal end configured to bluntly dissect between tissue layers as the blunt distal end is advanced there through.

In specific aspects of the transcutaneous delivery systems of the present invention, the ribbon stimulator may consist of an integrated structure comprising the elongate ribbon body, the one or more electrodes formed on the elongate ribbon body, and additional electrical circuitry on the ribbon body coupled to the one or more electrodes.

In other instances, the advancement may further comprise a handle attached to a proximal end of the elongate tool body, and may further comprise a finger grip distal to the handle.

In alternative instances, the advancement tool may further comprise a trigger mechanism for operating a mechanism to push the ribbon stimulator distally from the flat surface of the elongate tool body.

In still further instances, the flat surface of the advancement took may comprise a one-way coupling structure configured to mate with a one-way coupling structure on the ribbon stimulator, such that the coupling structures remain engaged and mated while the advancement tool is advanced through tissue and uncoupled when the advancement tool is retracted from tissue, leaving the ribbon stimulator in place.

In still further instances, the coupling structure may comprise interlocking elements which engage when pushed against each other and which disengage when pulled apart. In alternative instances, the coupling structure may comprise a bonding layer. In still further alternative instances, the coupling may comprise a sleeve.

In still further aspects of the transcutaneous delivery systems of the present invention, the ribbon stimulator may be pre-mounted on the advancement tool, often with a distal end of the ribbon stimulator located proximally behind a blunt distal end of the elongate tool body. Alternatively, in some instances, a distal end of the elongate tool body may be disposed proximally behind a blunt distal end of the ribbon stimulator, where the blunt distal end of the ribbon stimulator will then lead as the ribbon stimulator is advanced into tissue. In still further instances, the ribbon stimulator may comprise a support ring or other support element around at least a portion of a periphery of the ribbon stimulator.

In a third aspect, the present invention provides methods for fabricating a ribbon stimulator. Such methods typically comprise providing an electrode material, patterning the electrode material to form a plurality of electrode elements, supporting the electrode elements on a support surface, and applying a porous support material over the electrode elements while on the support structure to embed the electrode elements in the porous support material.

In some instances, the electrode material will be provided as a flat sheet, where the flat sheet may comprise a metal or other conductive material selected from the group consisting of platinum, platinum-iridium alloys, tantalum, stainless steel, and the like. In other instances, patterning the electrode material may comprise one or more metal working operations selected from a group consisting of piercing, blanking, lancing, perforating, trimming, shaving, parting-off and broaching, EDM, laser cutting, milling, chemical etching, powder metal molding, and the like.

In still other instances, the electrode elements formed by the fabrication methods of the present invention may comprise both electrode elements and connecting elements. In some instances, the electrode elements and the connecting elements may be formed in a single metal working operation, while in other instances, the electrode elements and the connecting elements may be formed in two or more different metal working operations.

In other embodiments of the fabrication methods of the present invention, supporting the electrode elements on a support surface comprises supporting the electrode elements on an electrode material strip. The electrode material strip may have pilot holes for providing alignment and subsequent forming operations. The plurality of electrode elements may be held in place on the electrode material strip by supporting elements which maintain a desired spacing between adjacent electrode elements during subsequent steps of the fabrication method. The electrode elements may be further joined by welding, brazing, crimping, or the like. The porous support material will typically be formed over the electrode elements while on the electrode material strip by electrospinning.

In a fourth aspect of the present invention, a ribbon stimulator comprises a support structure having a plurality of electrode elements formed on a surface thereof. The electrode elements are typically embedded in a porous substrate comprising layers having different mechanical properties, where the mechanical properties are selected so that the layers will separate in response to sheer forces such as those applied when the ribbon stimulator is being explanted. In such instances, the outer most layer of the porous substrate will be sacrificial so that it can be left behind as the remainder of the ribbon stimulator is removed. Typically, the outermost layer may be less elastic than an intermediate layer such that the intermediate layer will separate while sheer forces are applied, leaving the outermost layer in place attached to tissue.

In a fifth aspect, methods of the present invention comprise explanting the ribbon stimulator after it has been implanted in tissue. Explantation is achieved by pulling on a portion of the ribbon stimulator, typically using an explant tool, to apply a sheer force to the ribbon stimulator which causes a porous substrate of the ribbon stimulator to fail along a weakened plane between layers of the porous substrate. This way, a topmost, sacrificial layer of the porous substrate can remain embedded in tissue while the remainder of the ribbon stimulator may be removed from the tissue.

In yet another aspect of the present invention, an explantation tool for removing a ribbon implant from tissue comprises a shank having a handle on one end. A pair of flat jaws having blunt distal ends are attached to the other end of the shank and configured over closed one end of the ribbon implant to fixably engage that end and allow the tool to be pulled to remove the ribbon implant from the tissue.

Further aspects of the present invention are set forth in the following numbered clauses.
Clause 1: An transcutaneous delivery system comprising;
   a ribbon stimulator comprising at least one elongate ribbon body having one or more electrodes secured thereto; and
   an advancement tool comprising an elongate tool body having a flat surface for detachably receiving the elongate ribbon body of the ribbon stimulator;
   wherein at least one of the elongate tool body and the ribbon stimulator have a blunt distal end configured to bluntly dissect between tissue layers as the blunt distal end is advanced therethrough.
Clause 2: The transcutaneous delivery system of clause 1 wherein the ribbon stimulator consists of an integrated structure comprising the at least one elongate ribbon body, the one or more electrodes, and electrical circuitry on the ribbon body coupled to the one or more electrodes.
Clause 3: The transcutaneous delivery system of clause 1 wherein the advancement tool further comprises a handle attached to a proximal end of the elongate tool body.
Clause 4: The transcutaneous delivery system of clause 1 wherein the advancement tool further comprises a finger grip distal to the handle.
Clause 5: The transcutaneous delivery system of clause 1 wherein the advancement tool further comprises a trigger mechanism for pushing the ribbon stimulator distally from the flat surface of the elongate tool body.
Clause 6: The transcutaneous delivery system of clause 1 wherein the flat surface of the advancement tool comprises a one-way coupling structure configured to mate with a one-way coupling structure on the ribbon stimulator, wherein the coupling structures are configured to remain mated while the advancement tool is advanced through tissue and uncouple when the advancement tool is retracted from tissue.
Clause 7: The transcutaneous delivery system of clause 6 wherein the coupling structure comprises interlocking elements which engage when pushed against each other and disengage when pulled apart.
Clause 8: The transcutaneous delivery system of clause 6 wherein coupling structure comprises a bonding layer
Clause 9: The transcutaneous delivery system of clause 6 wherein coupling structure comprises a sleeve.
Clause 10: The transcutaneous delivery system of clause 1 wherein the ribbon stimulator is pre-mounted on the advancement tool.
Clause 11: The transcutaneous delivery system of clause 10 with a distal end of the ribbon stimulator placed proximally behind a blunt distal end of the elongate tool body.
Clause 12: The transcutaneous delivery system of clause 11 with a distal end of the elongate tool body placed proximally behind a blunt distal end of the ribbon stimulator.
Clause 13: The transcutaneous delivery system of clause 1 further comprising a support element around at least a portion of a periphery of the ribbon stimulator.
Clause 14: A method for fabricating a ribbon stimulator, said method comprising:
   providing electrode material;
   patterning the electrode material to form a plurality of electrode elements;
   supporting the electrode elements on a support surface; and
   applying a porous support material over the electrode elements on the support structure to embed the electrode elements in the porous support material.
Clause 15: The method of clause 14 wherein providing electrode material comprises providing a flat sheet of the electrode material.
Clause 16: The method of claim 15 wherein the flat sheet of electrode material comprises a metal selected from a group consisting of platinum, platinum-iridium alloys, tantalum, and stainless steel.
Clause 17: The method of clause 14 wherein patterning the electrode material comprises one or more metal working operations selected from a group consisting of piercing, blanking, lancing, perforating, trimming, shaving, parting off and broaching, EDM, laser cutting, milling, chemical etching, and powder metal molding.
Clause 18: The method of clause 14 wherein the plurality of electrode elements comprises electrode elements and connecting elements.
Clause 19: The method of clause 14 wherein the electrode elements and connecting elements are formed in a single metal working operation.
Clause 21: The method of clause 14 wherein electrode elements and connecting elements are formed in two or more metal working operations.
Clause 22: The method of clause 14 wherein supporting the electrode elements on a support surface comprises supporting the electrode elements on an electrode material strip.
Clause 23: The method of claim 22 wherein the electrode material strip has pilot holes for alignment in subsequent forming operations.
Clause 24: The method of claim 22 wherein the plurality of electrode elements are held in place on the electrode material strip by supporting elements which maintain a desired spacing between electrode elements.
Clause 25: The method of clause 14 further comprising joining some electrode elements welding, brazing or crimping.
Clause 26: The method of clause 14 wherein applying a porous support material over the electrode elements comprises electrospinning.
Clause 27: The method of clause 14 wherein the electrode material is provided as a flat sheet.
Clause 28: The method of clause 14 further comprising preparing surfaces of the electrode elements to promote adherence to the porous support material.
Clause 29: A ribbon stimulator comprising a support structure having a plurality of electrode elements formed on a surface thereof, wherein said electrode elements are embedded in a porous substrate comprising layers having different mechanical properties selected so that the layers will separate in response to shear forces applied to the ribbon stimulator during explantation.
Clause 30: The ribbon stimulator of claim 29, wherein an outermost layer of the porous substrate is less elastic than an intermediate layer such that the intermediate layer will separate while the outermost layer will remain attached to tissue.
Clause 31: A method for explanting a ribbon stimulator implanted in tissue, said method comprising pulling on a portion on the ribbon stimulator, wherein pulling applies a shear force to the ribbon stimulator which causes a porous substrate of the ribbon stimulator to fails along a weakened plane between layers of the porous substrate, wherein a sacrificial layer of the porous substrate remains embedded in the tissue while the remainder of the ribbon stimulator is removed from the tissue.
Clause 32: An explanation tool for removing a ribbon implant from tissue, said tool comprising:
   a shank;
   a handle at one end of the shank;
   a pair of flat jaws having blunt distal ends, wherein the jaws are configured to be closed over one end of the ribbon implant to fixedly engage that end and allow the tool to be pulled to remove the ribbon implant from tissue.
Clause 33: The explantation tool of claim 56, further comprising a slide assembly configured to be advanced over the jaws when closed and along the ribbon implant to break any tissue adhesion with the ribbon implant.
Clause 34: An implantable radiofrequency stimulator with preformed features which facilitate the placement, connection by galvanic or electromagnetic means and retention of the coupling feature of a separately implanted power source.

### Incorporation by Reference

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a flow diagram indicating steps of a method of delivering PNS therapy;

FIG. 2 is a flow diagram indicating steps of a method of improving headache disorders according to an embodiment of the present disclosure;

FIG. 3 is a flow diagram indicating steps of a method of improving headache disorders according to an embodiment of the present disclosure;

FIG. 4 is a schematic illustration of the back of the patient's head indicating a region to which a ribbon stimulator can be implanted according to an embodiment of the present disclosure;

FIG. 5 is a schematic illustration of a site in adipose tissue below the dermis and above galea (fascia) where a delivery tool can be advanced and a ribbon stimulator positioned according to an embodiment of the present disclosure;

FIG. 6 a schematic illustration of the target nerves;

FIG. 7 is a perspective view of a ribbon stimulator according to an embodiment of the present disclosure;

FIG. 8 is a perspective view of the delivery tool according to an embodiment of the present disclosure;

FIG. 9 is a perspective view of the distal end of the delivery tool according to an embodiment of the present disclosure;

FIGS. 10A-10E are perspective views of an alternate ribbon stimulator introducer;

FIG. 11 shows a process flow for a method of creating complex electrode structures for ribbon stimulators and other porous leads;

FIG. 12 is a representation of a “complex” electrode structure used to illustrate the manufacturing method;

FIGS. 13A-13C illustrates the principle of using a punch and die to form complex shapes in sheet material;

FIG. 14 shows the outline of the first piercing punch used to form the supported electrode structure;

FIG. 15 show a representation of the supported electrode structure after the first forming step;

FIG. 16 shows the supported electrode structure with a porous covering;

FIG. 17 shows the outline of the second blanking and piercing punch used to remove the electrode supports and surround material;

FIG. 18 shows the completed complex electrode structure ready to enmeshing in a porous substrate;

FIGS. 19A and 19B show a porous substrate with a composite structure that can be used to assist in removal of an implanted device;

FIG. 20 shows a ribbon stimulator configured as an RF stimulator with an external RF power source;

FIG. 21 shows a ribbon stimulator configured with an implantable power source;

FIGS. 22A-22F shows a method of converting a ribbon stimulator with external RF power supply to one with an implantable power source;

FIGS. 23A-23D shows a tool and method of removing a ribbon stimulator from tissue;

FIG. 24 is a flow diagram indicating steps of a general method of improving a range of disorders according to an embodiment of the present disclosure;

FIGS. 25A-25C show a one-step method of implanting a ribbon stimulator using a specialized tool;

FIGS. 26A and 26B show a method of push implanting a ribbon stimulator using one or more permanent features of the ribbon stimulator;

FIGS. 27A-27C shows a method of push implanting a ribbon stimulator using one or more temporary features of the ribbon stimulator;

FIG. 28 shows a ribbon stimulator with one or more temporary features which support a pull implantation method; and

FIG. 29 is a flow diagram indicating steps of a method of removing an implant.

### DETAILED DESCRIPTION OF THE INVENTION

The present disclosure generally relates to methods and devices for improving chronic pain disorders in general and headache disorders in particular in a patient suffering therefrom by electrically modulating neural tissue in a minimally invasive fashion using an electrical ribbon stimulator. A “ribbon stimulator” as used herein has a width of greater than 1 mm and less than approximately 10 millimeters (mm), a thickness of greater than 0.5 mm and less than approximately 2 mm and a length of greater than 10 mm and less than approximately 150 mm.

As used herein with respect to a described element, the terms “a,” “an,” and “the” include at least one or more of the described element unless otherwise indicated. Further, the terms “or” and “and” refer to “and/or” unless otherwise indicated. It will be understood that when an element is referred to as being “over,” “on,” “attached” to, “connected” to, “coupled” with, “contacting,” “in communication with,” etc., another element, it can be directly over, on, attached to, connected to, coupled with, contacting, or in communication with the other element or intervening elements may also be present. In contrast, when an element is referred to as being “directly over,” “directly on,” “directly attached” to, “directly connected” to, “directly coupled” with, “directly contacting,” or in “direct communication” with another element, there are no intervening elements present. An element that is disposed “adjacent” another element may have portions that overlap or underlie the adjacent element. By “substantially” is meant that the shape, configuration, or orientation of the element need not have the mathematically exact described shape, configuration or orientation but can have a shape, configuration or orientation that is recognizable by one skilled in the art as generally or approximately having the described shape, configuration, or orientation.

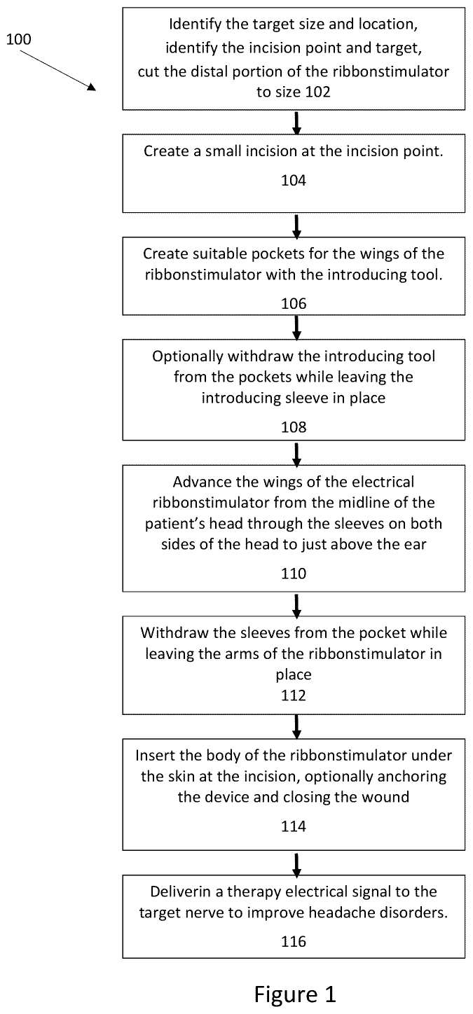

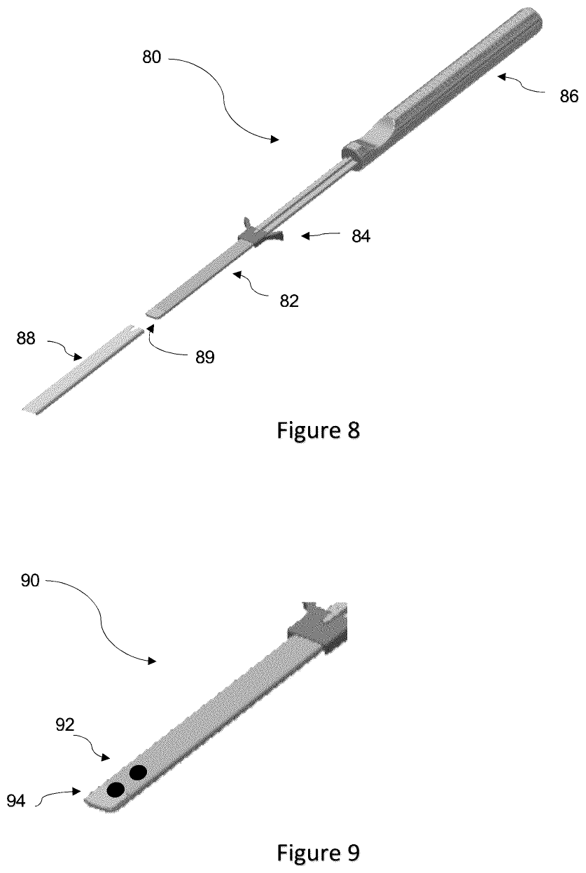

Referring to FIG. 1, in an embodiment, a method (100) of delivering PNS can comprise identifying the target site and location, identifying the incision point and target, cutting the distal portion of the ribbon stimulator to size (step 102) creating a small incision at the incision point (step 104), creating suitable pocket or pockets, using the introducing tool (FIG. 8 80) (step 106), withdrawing the introducing tool while optionally leaving the introducing sleeve(s) (82) in the pocket(s) to hold the pocket open (step 108) and inserting the electrode ribbon(s) of the ribbon stimulator through the introducing sleeve(s) (step 110). Once the electrode ribbon(s) is (are) in place the introducing sleeve(s) is (are) withdrawn (step 112) and the body of the ribbon stimulator is inserted under the skin at the incision before closing the wound with any suitable method (step 114). Method 100 can further include positioning the ribbon stimulator at a target implant site adjacent to a target nerve associated with the condition to be treated (step 116). Method 100 can further comprise anchoring the ribbon stimulator to the target implant site (step 114). Once the ribbon stimulator has been anchored to the target implant site, method 100 can comprise delivering a therapeutic electrical signal to the target nerve to deliver the target therapy (step 116). The therapeutic electrical signal may modulate the target nerve by increasing or decreasing neuronal activity. As such, the therapy electrical signal can be an excitatory or inhibitory stimulation signal or a combination thereof. The therapy electrical signal may also mask, alter, override, or restore neuronal activity.

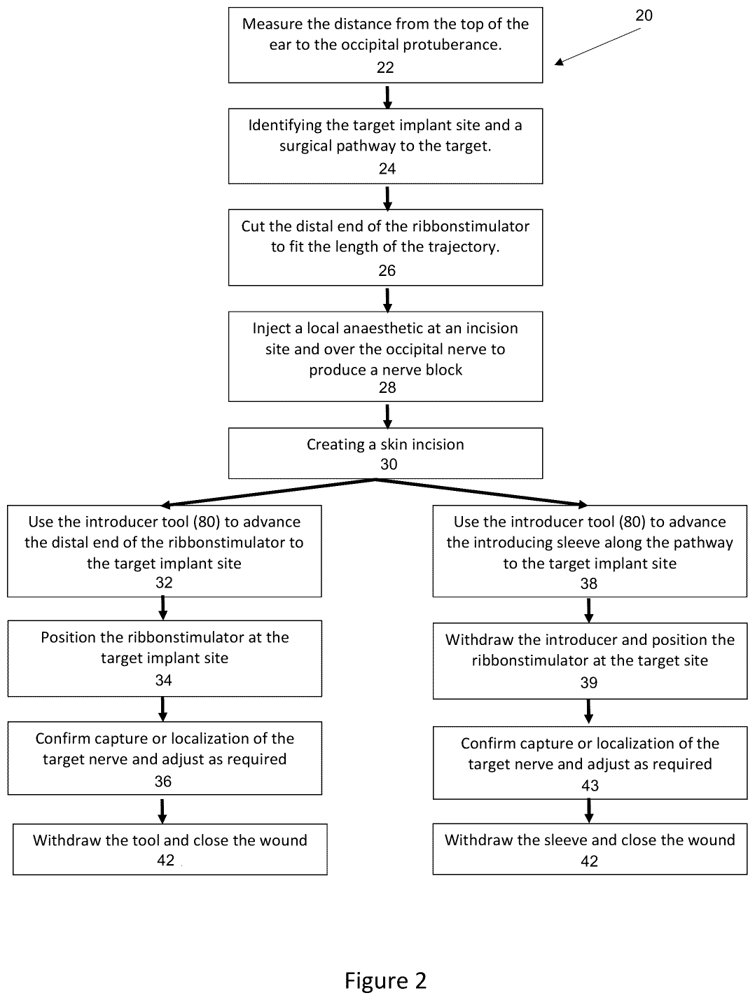

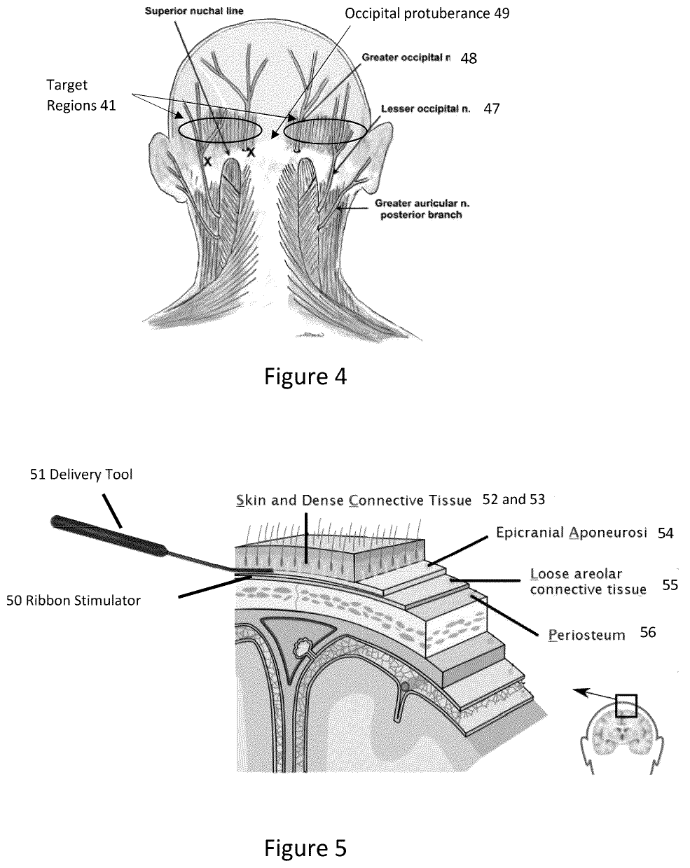

Referring to FIG. 2, in an embodiment, a method (200) of improving headache disorders can comprise locating the surgical implant site with reference to anatomical features such as the ear, the cranial midline and/or the occipital protuberance (step 202), identifying the target implant site and a surgical pathway to the target (step 204), cutting the distal end of the ribbon stimulator to fit the length of the trajectory (step 206), injecting a local anesthetic at an incision site and over the occipital nerve to produce a nerve block (step 208), creating a skin incision, (step 210) using the introducer tool (80) to advance the distal end of the ribbon stimulator along the surgical pathway to the target implant site (step 212), positioning the ribbon stimulator at the target implant site (step 214), confirming capture or localization of the target nerve and readjusting the location of the ribbon stimulator as necessary, (step 216) withdrawing the introducing tool and closing the wound (step 222), or alternatively leaving the introducing sleeve(s) (52) in the pocket(s) to hold the pocket(s) open (step 218) and inserting the electrode ribbon(s) of the ribbon stimulator through the introducing sleeve(s) (step 220) Once the electrode ribbon(s) is (are) in place the introducing sleeve(s) is (are) withdrawn and the body of the ribbon stimulator is inserted under the skin at the incision before closing the wound with any suitable method (step 222). The wing(s) of the ribbon stimulator can be advanced to region 41 as illustrated in FIG. 4. Method 200 can further include positioning the ribbon stimulator at a target implant site adjacent to a target nerve associated with headache disorders (step 214).

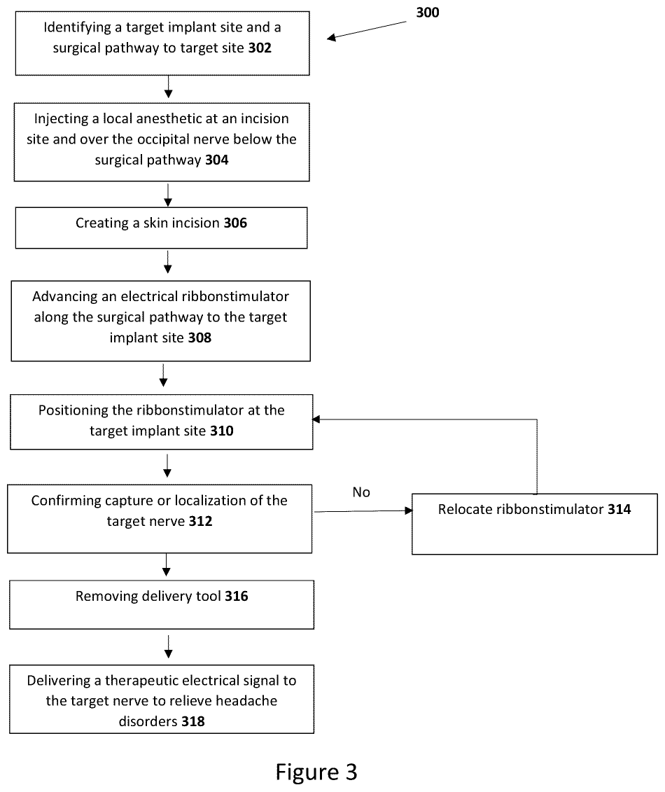

In certain embodiments, the target implant site is above the fascia, epicranial aponeurosis or galea and below the dermis illustrated in FIG. 5. Method 200 can further comprise anchoring the ribbon stimulator to the target implant site (step 220). Once the ribbon stimulator has been anchored to the target implant site, method 200 can comprise delivering a therapy electrical signal to the target nerve(s) to improve headache disorders, chronic back pain disorders or other disorders. The therapy electrical signal can modulate the target nerve by increasing or decreasing neuronal activity. As such, the therapy electrical signal can be an excitatory or inhibitory stimulation signal or a combination thereof. The therapy electrical signal may also mask, alter, override, or restore neuronal activity. Referring to FIG. 3, in an additional or alternative embodiment, a method of treating disorders by neuromodulation (300) can comprise identifying a target implant site to implant the ribbon stimulator and a surgical pathway to reach the target implant site (step 302). The target implant site is adjacent to a target nerve (or nerves) associated with headaches, back pain or other disorder. The target implant site can be determined, for example, using ultrasound, external skin electrodes, anatomical landmarks, or other imaging or stimulating techniques. Visualization or localization of the target nerve as an initial step can be used to guide a clinician in selecting an incision site and the length of the ribbon electrode. After locating the target implant site and the surgical pathway to the target implant site and cutting the ribbon electrode to size, method 300 can comprise injecting a local anesthetic at an incision site and along the surgical pathway or over the occipital nerve to create a local nerve block (step 304). This can be done by inserting a syringe to the implant target location along the pathway to the implant target location and slowly injecting anesthetic while removing the syringe, or with a syringe with multiple exit ports along the length that distribute anesthetic along the entire pathway.

At step 306, a skin incision can be created. In particular and with additional reference to FIG. 5, ribbon stimulator 50 can be inserted through skin tissue by incising the outer skin layers 52 using, for example, a standard scalpel or an incising edge or blade of a delivery tool. The incision is preferably minimal in size to tightly accommodate ribbon stimulator 50. For example, the length of the skin incision can be approximately equal to the width of an incising end of a delivery tool to provide an incision just large enough to insert ribbon stimulator 50) or the distal end of delivery tool 51 used to deploy ribbon stimulator 50.

At step 308, method 300 can comprise creating a suitable pocket or pockets for the electrode ribbon(s) of the ribbon stimulator with an introducing tool then withdrawing tool and possibly leaving an introducing sleeve in place to hold the pocket(s) open (step 310). The ribbon electrode(s) is (are) then advanced along the surgical pathway to the target implant site. As illustrated in FIG. 5, ribbon stimulator 50 can be inserted and advanced subdermally via delivery tool 52 to the target implant site. The delivery tool can have a flexible tip and/or a blunt tip (as described in more detail below) so that it can be advanced at a shallow angle until deflected by the galea (fascia) layer 54 and thereby to the target implant site that is above galea (fascia) layer 54 as schematically illustrated in FIG. 5. Such a flexible and/or blunt tip of the delivery tool can allow placement of the ribbon stimulator as close as possible to the galea (fascia) while remaining within the safest tissue region. The incision can be mechanically pinched together and closed using a stitch, clip, staple or other closure device.

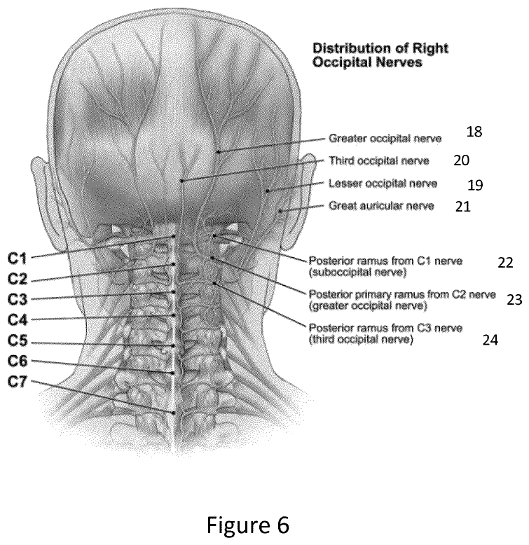

With further reference to FIG. 5, method 300 can further include positioning ribbon stimulator 50) at the target implant site (step 310). As mentioned above, the target nerve is a nerve associated with headache prevention. In certain embodiments, the target implant site is above galea (fascia) 54 adjacent to a target nerve, such as target nerve 48. With reference to FIG. 6, exemplary target nerves are greater occipital nerve 68, lesser occipital nerve 69, third occipital nerve 60, greater auricular nerve 61, posterior ramus from C1 62, posterior ramus from C2 23 posterior ramus from C3 64, or combinations thereof. Illustrated in FIG. 6. The target nerve can be two or more nerves and their branches and as used herein “a target nerve” can include a plurality of nerves. In certain embodiment, a target nerve is the greater occipital nerve and the lesser occipital nerve.

Regarding step 312, capture or localization of the target nerve can be confirmed.

Referring again to FIG. 3, at step 314, allow for relocation of the ribbon stimulator if the nerve is not adequately captured. The ribbon stimulator can be positioned parallel, perpendicular or at any angle to the target nerve.

At step 316, sufficient nerve capture confirmation is performed, as described in step 312. Nerve capture confirmation can be achieved by delivering test stimulation pulses and measuring and/or observing a stimulation response. If the proper stimulation location is verified, the ribbon stimulator can be released and the delivery tool can be removed (step 316). Alternatively, if the stimulation location and anchoring strength are not adequate the delivery tool can reposition the ribbon stimulator (step 316). Steps 310 through 314 can be performed until sufficient stimulation and anchoring are achieved.

Once sufficient stimulation and anchoring are achieved at step 312, the ribbon stimulator can be released from the delivery tool (as described in more detail below) and the delivery tool can be removed (step 316). In certain embodiments, prior to release of the ribbon stimulator, the ribbon stimulator can be programmed to deliver a stimulation signal having pre-determined parameters, such as a pre-determined intensity, that are deemed to have therapeutic benefits. The patient's response to such stimulation can be observed or detected for any painful or uncomfortable response. Gauging the sensation perceived by the patient before releasing the ribbon stimulator from the delivery tool can increase the probability of prescribed therapy compliance and decrease adverse effects due to stimulation. At step 318, the ribbon stimulator can deliver a therapeutic electrical signal to the target nerve to improve headache disorders.

The above described methods are exemplary and other methods for implanting a ribbon stimulator to deliver a therapy signal to a target nerve to improve headache disorders can include combinations and sub-combinations of the above-described steps, including the elimination or addition of certain steps.

In order to predict the potential efficacy of treating a pain or headache condition with the ribbon stimulator in a given patient, a temporary trial procedure may be performed before a permanent implant is attempted. This trial procedure might utilize an electrode ribbon that is inserted under the skin and connected to an external trial stimulator. The electrode ribbon for the trial procedure might be a two-wire electrode substantially equivalent in function to the ribbon electrodes described herein or it may be a single wire electrode, which could be introduced through a standard needle with a single puncture. The single wire trial electrode could be used alone, to deliver monopolar stimulation or paired with an externalized electrode, placed on the surface of the skin to deliver bipolar stimulation.

In certain embodiments, the trial electrode(s) described may be coated with a material that has mechanical properties similar to the tissue at the target implant site, to mimic the permanent electrode, (possibly) reduce tissue reaction, decrease the likelihood of known adverse events such as lead migration or infection, and/or foster more rapid nerve recruitment. Exemplary materials include polymeric materials with elastic properties or thermal plastics comprising urethane, aromatic polyurethane, silicones, polyethers, polycarbonates, polytetrafluoroethylene, elastane, or combinations thereof.

In certain methods of relieving headache disorders as disclosed herein, a ribbon stimulator can be implanted in a target implant site that is above galea (fascia) adjacent to a target nerve associated with headache disorders. Compared to medications for treating headaches which exert their effect globally certain methods as disclosed herein involve implanting a ribbon stimulator directly adjacent to the target nerve and therefore such procedures have a greater probability of activating the target nerve. However, methods and devices disclosed herein can deliver an efficacious electrical therapy signal to the target nerve and can also securely and safely fixate the ribbon stimulator to looser connective or adipose tissue of the target implant site that is above galea (fascia).

Regarding delivering an efficacious electrical therapy signal, electrical current delivered by the ribbon stimulator can be steered to shape the stimulation field to ensure appropriate nerve capture. For example, a ribbon stimulator can be used that has independently programmable electrodes that can each be activated, deactivated, programmed to deliver a certain percentage of electrical current, and/or have independent current sources (stimulation channels) to customize, shape and steer the electrical current. Independently programmable electrodes also allow the modulation to be directional in nature applying an activation signal to only certain regions while sparing modulation to others. Such directional electrodes allow for precise selective modulation of the target nerve as well as allow steering of the electrical signal. Independently programmable electrodes also allow simultaneous or sequential delivery of electrical signals to one or more target nerves with each electrical signal having stimulation parameters, such as frequency, amplitude, pulse width, specific to the target nerve to maximize therapy. A set of specific values for each stimulation parameter can constitute a program (for example 2 Hz. 10 mA. 50 ms respectively). Further, the ribbon stimulator can be programmed to deliver at least two independent stimulation programs to the target nerve.

In addition to modulating the direction of the electrical signal, the degree of activation that each electrode delivers can be adjusted. For example, the pulsing parameters of electrodes may be adjusted to initiate, stop, increase, or decrease the pole combinations, energy, amplitude, pulse width, waveform shape, frequency, and/or voltage or other pulsing parameter to adjust the degree of modulation delivered thereby. Additionally, the shape of the electric field can vary corresponding to the power applied, the number and arrangement of electrodes, and particular shapes and sizes chosen for the electrodes. For example, the electrodes can be long wires or can be segmented into short section by masking off some of the wire or adding additional wires. Electrodes can also be flat metal arranged in various ways to allow for focused stimulation.

Furthermore, each electrode may be selectively powered as an anode, cathode. The electrode function may be provided by conductive elements in the lead structure, by the case of the stimulator element or by an external patch electrode attached to the skin. Such external electrode may be physically attached to the power transmitting device or be placed remote from the transmitter. For example, a ribbon stimulator can have any combination of cathodes and anodes (as long as there is at least one cathode and at least one anode) thereby providing different shaped current fields. Alternatively, a ribbon stimulator can be programmed such that only one pair of electrodes is active at any given time, limited to either a top pair of electrodes or a bottom pair of electrodes. Further, the electrodes can be sufficiently spaced apart to allow independent current settings for each of the electrodes of the ribbon stimulator. In certain embodiments, electrodes are positioned on the two widest portions of a ribbon stimulator have a top surface and a bottom surface. The electrodes on the bottom surface will be facing towards deep target nerves and the electrodes on the top surface will be facing towards more superficial target nerves If the electrodes are placed in other locations one set of electrodes will stimulate in one direct and the other in the opposite directions. In certain embodiments, a ribbon stimulator can include an Application Specific Integrated Circuit (ASIC) to provide current steering.

In certain embodiments, a ribbon stimulator has two electrodes in total, one electrode positioned on each side of the porous substrate. In one embodiment, each stimulation pulse is between an anode and cathode.

In other embodiments the ribbon stimulator may have more than two electrodes positioned variously within the porous substrate. In these embodiments two or more electrodes can be configured, possibly dynamically, at act as anodes and cathodes and stimulation pulses may be between any anode and cathode as may be required for the effective delivery of therapy.

Such steps of controlling the direction and shape of the electrical signal applied to the target nerve can be performed after implantation of the ribbon stimulator.

Ribbon stimulators as disclosed herein can be part of a system including a remote pulse generator (not shown) that is in electrical communication with an electrode of the ribbon stimulator and is configured to produce one or more electrical signals. Alternatively, ribbon stimulators can comprise an integral pulse generator. In addition, ribbon stimulators can include an integral battery that is rechargeable by inductive coupling or can be part of a system that includes a remote battery operably coupled to the ribbon stimulator. In other words, a ribbon stimulator may be powered by bringing a power source external to the patient's body into contact with the patient's skin or may include an integral power source. As such, a pulse generator or battery may be positioned in any suitable location, such as adjacent to the ribbon stimulator (e.g., implanted adjacent to the ribbon stimulator), integral with the ribbon stimulator, or at a remote site in or on the patient's body.

In some instances, the ribbon stimulator can include its own power source, e.g., which is capable of obtaining sufficient power for operation from surrounding tissues in the patient's body. Internal power sources can obtain sufficient energy, for example, from muscle movements and other source of body energy generation that can be harnessed via a capacitor or a balloon device that harnesses the energy, for example, so that an internal battery is not needed.

Ribbon stimulators can be pre-programmed with desired stimulation parameters. Stimulation parameters can be controllable so that an electrical signal may be remotely modulated to desired settings without removal of the ribbon stimulator from the target implant site. Remote control may be performed, e.g., using conventional telemetry with an implanted pulse generator and battery, an implanted radiofrequency receiver coupled to an external transmitter, and the like. In some instances, some or all parameters of the ribbon stimulator may be controllable by the patient, e.g., without supervision by a physician. In other instances, some or all parameters of the ribbon stimulator may be automatically controllable by a programmer or controller. In this instance external triggers such as atmospheric pressure or activity monitors may provide an input to the ribbon stimulator to turn on or off the stimulation. A controller can be embodied as software on a multi-purpose external device, such as, for example, a PC, a cell phone, a PDA type device, or tablet.

In certain embodiments as disclosed herein, the controller unit may track or collect information which, for example, relates to migraine event triggers and use that information to automatically control the delivery of therapy from the ribbon stimulator. Information used to control may be gathered manually, for example users may enter the occurrence of a potential trigger such as consumption of alcohol, the system may collect biological information automatically, either from wearables such as galvanic skin resistance sensors to infer stress, or other devices such as sleep monitor data, or it may be gathered from external sources such as weather data (since some aspects of weather patterns such as barometric pressure have been reported as possible migraine event triggers). Such embodiments may use an algorithmic approach to identifying relationships between triggers and migraine events and use that information to automatically control various aspects of the ribbon stimulator including but not limited to stimulation on or off, stimulation amplitude, stimulation frequency or the choice of stimulation electrodes.

Certain embodiments as disclosed herein include closed-loop systems. For example, the patient controller could collect information such as barometric pressure that could be a trigger to headaches. The device would then be automatically controlled, using settings previously programmed by the clinician, to prevent the onset of a headache. Other signals that may be used to control the device that would be collected by the controller include but not limited to patient activity, sleep, food, and drink. In addition to data collected in the controller biological signals may be used by applying sensors directly to the patient using the RF transmitter or additional wearable straps such as EMG or ENG. The transmitter or physician or programmer can have a plug-in sensor to sense EMG activity, sense ENG activity, or measure trigeminal somatosensory evoked potentials (TSEPs), evoked muscle action potentials (EMAPs) or electrically evoked compound action potentials (ECAPs) to determine the minimal threshold of stimulation needed to achieve a therapeutic effect. Identifying the minimal threshold needed for stimulation avoids or minimizes pain for the patient. Such a feature could also be used for troubleshooting, programming, patient feedback etc. Other art stimulates at the highest tolerable level, since they are open loop, with the understanding that such stimulation will result in the highest probability of being efficacious. Also, other art relies on physiological responses such as twitches or painful muscle contraction to evaluate the programming settings.

Because a ribbon stimulator, according to certain embodiments of the present disclosure, may be implanted at a site that is above galea (fascia) in a region with minimal tissue, the ribbon stimulator must be thin and flexible. To address such a concern, ribbon stimulators as disclosed herein can have a flat or cylindrical, elongated, low profile configuration. The majority of the ribbon stimulator can be fabricated from or coated with a material so that the ribbon stimulator is “body compliant” and has mechanical properties similar to the tissue at the target implant site. Exemplary materials include polymeric materials with elastic properties or thermal plastics comprising urethane, aromatic polyurethane, silicones, polyethers, polycarbonates, polytetrafluoroethylene, elastane, or combinations thereof.

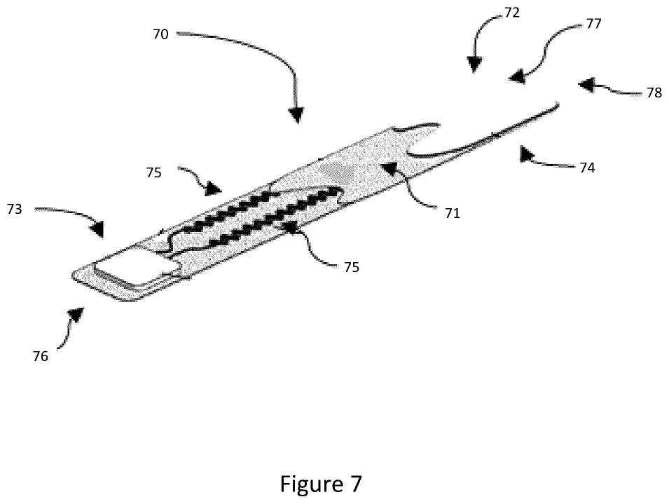

Referring to FIG. 7, in an embodiment, a ribbon stimulator 70 comprises a ribbon stimulator body 71 having a top surface 72, a bottom surface 74, a proximal end 76 and a distal end 78. Ribbon stimulator body 71 can include an enclosure 73 comprising electrical circuitry that is in electrical communication with at least one independently programmable electrode 75 The body 71 of the ribbon stimulator is made from a porous substrate which incases the enclosure 73 and one or more electrodes 75. The on top surface 72 or on bottom surface 74 may contain an insulating material 77. Although FIG. 7 illustrates the enclosure at the distal end of the ribbon stimulator body, the enclosure can be located at the proximal end or anywhere between the proximal and distal end. In certain embodiments the ribbon stimulator may have one stimulation “arm” (as shown in FIG. 7) and in other embodiments it may have two or more arms configured to form a single linear device or with the arms disposed at defined angles to each other. The electrical circuitry within enclosure 73 can include microprocessors under the control of a suitable software program. The electrical circuitry can include other components such as an analog-to-digital converter, etc.

In certain embodiments, as depicted in FIG. 7, ribbon stimulator body 71 comprises two independently programmable electrodes 75a and 75b imbedded in the porous body 71 are separated by a distance of at least two millimeters. The porous material of the ribbon stimulator body is designed to emulate the body's extra-cellular matrix (ECM), which creates a self-anchoring property eliminating the need for a physical anchor. Furthermore, in certain application, the ribbon material can be sutured or fixed to a tissue to create increased fixation.

Although the above embodiments describe self-anchoring device the porous body can be made more or less porous creating areas of greater or less fixation. The ribbon stimulator itself and can contain biodegradable materials which, upon degradation, create an anchor point.

A delivery tool used to implant a ribbon stimulator can include features to facilitate tunneling through skin to an implant site without penetrating galea (fascia) or blood vessels. Referring to FIG. 8 a delivery tool 80 can have a blunt tip 89 that provides for blunt dissection of subdermal tissue prior to reaching galea (fascia) to avoid penetration of galea (fascia). An insertion portion 82 of the tool can be made of a flexible metal over molded with silicone or TPU. A handle 86 and finger grip 84 will allow easy manipulation by the surgeon. A flexible thin walled plastic sleeve 88 allows easy insertion of the ribbon stimulator.

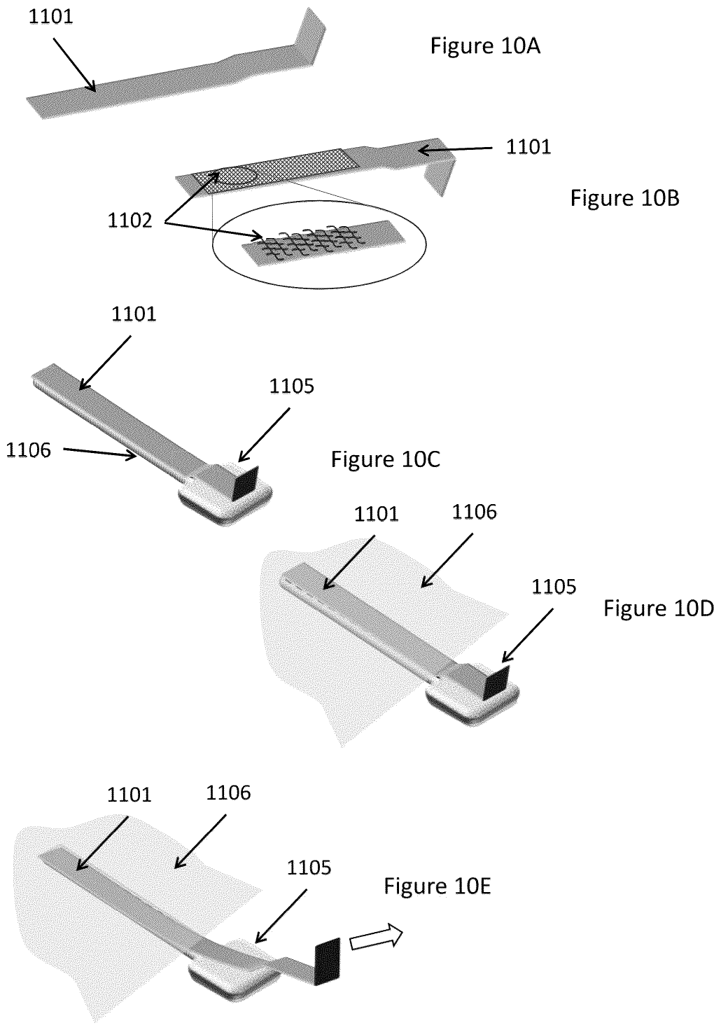

It will be understood that a typical ribbon stimulator is quite compliant and advancing the ribbon stimulator into the tissue may be difficult. With reference to FIG. 10, in some embodiments of this invention a stiff but flexible applicator 1101 (FIG. 10A) made from a suitable biocompatible material, is temporarily bonded to the porous coating of the electrode array of the ribbon stimulator 1105 to add structure and stiffness to said arms (FIG. 10C). This temporary bond may be made by means of coupling structure 1102 integrated onto the underside of the applicator 101 (FIG. 10B) configured such that force in one direction will tend to cause the coupling structure to couple with the porous coating of the ribbon stimulator electrode array 1106, while force in the opposite direction will tend to cause the coupling structure to decouple with the ribbon stimulator array 1106. For example, as illustrated in FIG. 10B, the coupling structure may comprise interlocking elements which engage when pushed against each other and disengage when pulled apart. With reference to FIG. 10C, the applicator, 1101 can be coupled to a ribbon stimulator electrode array 1106 to provide temporary stiffening of the electrode array 1106. With reference to FIG. 10D, the combined ribbon stimulator electrode array 106 and applicator 101 can be easily advanced through the tissue 1108 in which the ribbon stimulator is implanted, once the ribbon stimulator electrode array 1106, is in place, the applicator, 1101, is withdrawn causing it to decouple from the electrode array 1106 and leave the ribbon stimulator electrode array, 1106, flat and in the correct position. It will be understood that the applicator 1101 described herein could also be used in conjunction with the insertion sleeve 88 described above. It will be further understood that other methods of achieving a temporary bond between the applicator 1101 and the electrode array 1106 exist. One example would be a temperature sensitive glue that achieves adhesion between the introducer 1101 and the electrode array 1106 at room temperature but which loses adhesion when the assembly is implanted and increases in temperature.

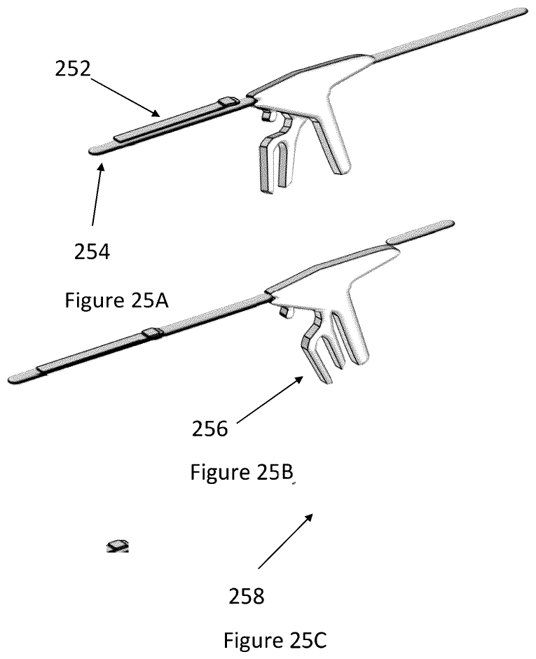

The mechanical properties of the ribbon stimulator coupled with the subcutaneous placement required in many applications allow for a range of different approaches to inserting the device. According to one aspect of this invention the ribbon stimulator may be mounted on transported subcutaneously by a tool which combines the blunt dissection function and the device transport function. With reference to FIGS. 25A-25C, the ribbon stimulator, 252, is mounted on a combined carrier and moderately compliant blunt dissection element 254 (FIG. 25A). A small incision slightly greater than the width of the tool is made in the appropriate place, and the combined assembly is placed at the incision and advanced or pushed, perhaps by means of a trigger mechanism 256 (FIG. 25B) or other means, such that the blunt tip of the compliant dissection element, 254, forms a pocket between the appropriate two layers of tissue. When the ribbon stimulator, 252, is advanced to the correct location it is disengaged from the compliant dissection element, 254 (FIG. 25C), perhaps by means of a trigger mechanism, 258, or other means, the compliant dissection element, 254, is withdrawn, leaving the ribbon stimulator, 252, in place. The ribbon stimulator, 252, may be held in place during this last step, for example, by the implanting physician applying light pressure to the device through the patient's skin.

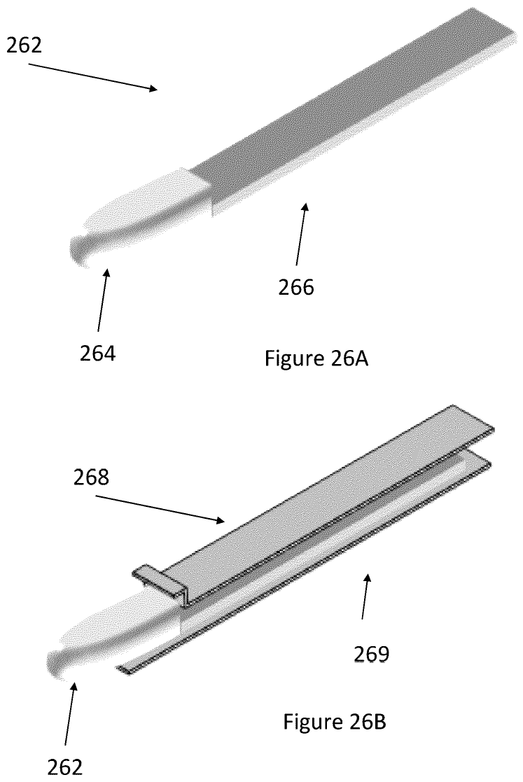

According to another aspect of this invention, and with reference to FIG. 26A, the ribbon stimulator, 262, which comprises a stimulation element, 264, and one or more ribbon electrodes, 266, can be so formed that some permanent feature or features of the ribbon stimulator, 262, are used in the implantation process. As an example of this aspect of the invention it will be noted that the stimulation element, 264, in FIGS. 26A and 26B has rounded or blunt face distal to the ribbon electrode, 266, and a square face proximal to the to the ribbon electrode, 266. With reference to FIG. 26B, an implantation tool may then be formed which may clamp the ribbon stimulator, 262, between a force transfer element, 268 and a support element, 269. When the ribbon stimulator, 262, is so clamped, the rounded or blunt face of the stimulation element, 264, may be pushed into the tissue with the rounded or blunt face used as the blunt dissection tool required to create the pocket in the tissue. Pressure applied by the implanting physician to the insertion tool is transferred to the square face proximal to the to the ribbon electrode, 266, by means of the force transfer element, 268. When the ribbon stimulator, 262, is advanced to the correct location the clamping action of the force transfer element, 268, is released, perhaps by means of a trigger mechanism or other means, and the tool is withdrawn, leaving the ribbon stimulator, 262, in place. The ribbon stimulator, 262, may be held in place during this last step, for example, by the implanting physician applying light pressure to the device through the patient's skin.

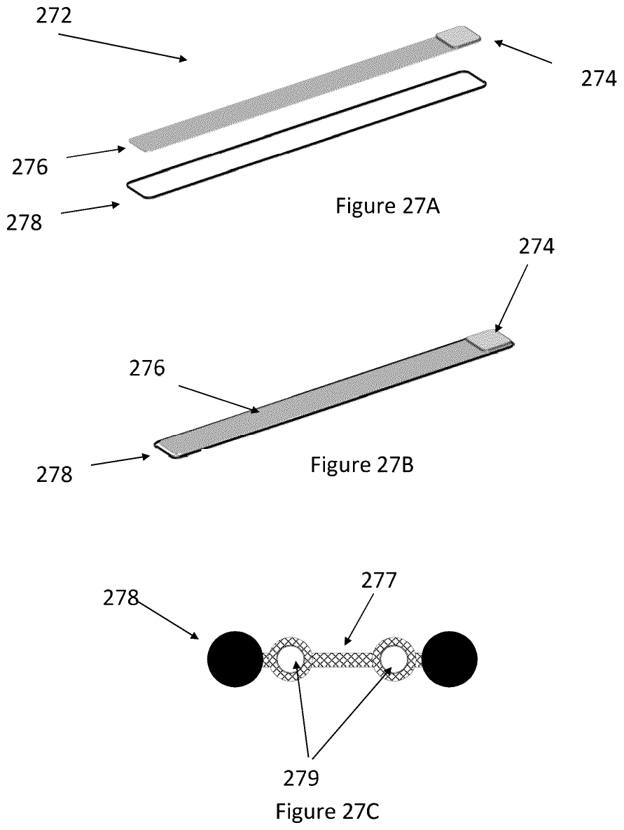

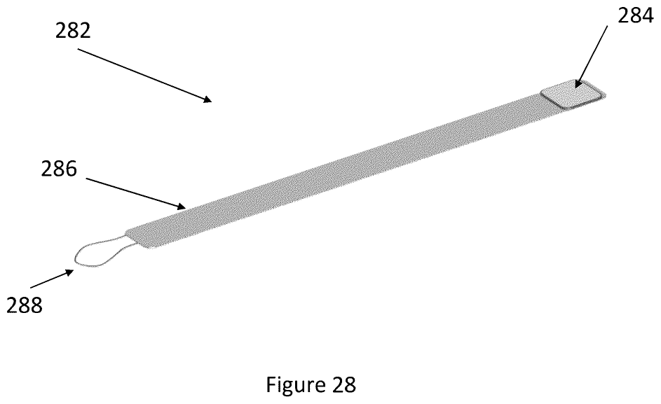

According to another aspect of this invention and with reference to FIGS. 27A-27C, the ribbon stimulator, 272, which comprises a stimulation element, 274, and one or more ribbon electrodes, 276, can be so formed that some temporary feature or features of the ribbon stimulator, 272, are used in the implantation process. As an example of this aspect of the invention the ribbon stimulator, 272, shown in FIGS. 27A and 27B has an additional component herein called a support element such as a ring, 278. This support ring, 278, shown displaced for clarity in FIG. 27A and in place in FIG. 27B, for formed from a moderately compliant, biocompatible and bioresorbable polymer such as polylactic acid (PLA). The support ring, 278, is molded into the ribbon stimulator during the manufacturing, and has the effect of stiffening the ribbon stimulator, 272, so it may be conveniently gripped and pushed into a suitable pocket in the tissue. As can be seen in the cross-section view of the lead part of the ribbon stimulator, 272, the support ring, 278, is molded onto the porous matrix, 277, of the ribbon lead, but clear of the electrode elements, 279 (FIG. 27C). The support ring, 278, therefore provides temporary stiffening for the whole device without interfering with its electrical operation. Those skilled in the art will understand there are other arrangements of a temporary device features that can be used to facilitate implantation by pushing and these arrangements are also claimed in this invention.

According to a further aspect of this invention, implantation can also be readily achieved by a pulling method. With reference to 28, the ribbon stimulator, 282, is fabricated with a stimulation element, 284, and one or more ribbon electrodes, 286, and a temporary attachment element structure, 288, made from a suitable biocompatible and bioresorbable polymer such as polylactic acid (PLA). At implantation an incision is made at each end of the surgical path for the device and a suitable pocket made between the two incisions. A thin, hooked stylet or similar surgical tool is then passed through the pocket from one end to the other, hooked to the temporary such as a loop structure, 288, and the ribbon stimulator, 282, is pulled into the pocket. At the completion of the procedure each incision is then sutured shut. Those skilled in the art will understand there are other arrangements of a temporary device features that can be used to facilitate implantation by pushing and these arrangements are also claimed in this invention.

Those skilled in the art will understand there a ribbon stimulator may also be constructed with one or more permanent device features that can be used to facilitate implantation by pulling and these arrangements are also claimed in this invention.

Ribbon stimulators and delivery tools can also include features that allow a clinician to detect the location of the tool as the ribbon stimulator is inserted into tissue. Such features may include mechanical or electrical sensors. For example, referring to FIG. 9, a delivery tool 90 can include sensing electrodes 92 and stimulation electrodes 94 to provide for real-time monitoring to determine the location of tool. In embodiments where the ribbon stimulator is implanted near the deep fascia, the device may measure the impedance between electrode pairs. Impedance generally decreases from the skin to deep fascia. As such, typical fascia tissue has lower impedance than tissue above deep fascia. Therefore, a clinician can detect when the electrodes are in contact with fascia. In other words, while the delivery tool is advanced along the tissue to an implant site, impedance can be monitored to provide feedback so that the delivery tool maintains contact with the tissue along the surgical pathway but does not penetrate into fascia or galea. Such feedback can be provided to the clinician during insertion to detect the galea, to ensure that the delivery tool reaches the interface between tissue above galea and the galea layer, and allow for a change of insertion angle to prevent puncture of galea (fascia). Further, during advancement of the delivery tool and ribbon stimulator, real time monitoring is possible to ensure continued contact with tissue above the galea (fascia). Impedance monitoring electrodes can also be used for stimulation and/or sensing during implant procedures for locating or detecting the target nerve or for target nerve capture confirmation testing to determine the target implant site.

In another embodiment the electrodes 92 and 94 may be used to transmit a stimulation signal and measure any compound action potential that stimulation evokes. By monitoring the measured response to stimulation as the ribbon stimulator is advanced through the tissue, the surgeon may confirm that the device has passed near to neural tissue or not.

As described above, a delivery tool can include stimulation and/or sensing electrodes to provide an electrical signal during the implant procedure, while monitoring for sensed nerve activation, such as EMG or ENG signals, for example. Nerve activation can be monitored in other ways as well. The stimulation or sensing electrodes can be on multiple sides of the delivery tool to allow for stimulation and nerve capture sensing of more than one nerve. In addition, sensing can be done from external electrodes placed on the skin, such as EMG or ENG electrodes. The delivery tool can include visual feedback indications relating to target nerve activation such as LED indicators Such localization features that are utilized while the delivery tool is advanced provide feedback for target ribbon stimulator placement adjacent a target nerve. Further, multiple feedback signals can be obtained if targeting more than one nerve. Other nerve activities or potentials that can be monitored during placement include trigeminal somatosensory evoked potentials (TSEPs), evoked muscle action potentials (EMAPs), and electrically evoked compound action potentials (ECAPs).

The ribbon stimulators described herein may have simple or arbitrarily complex electrode arrays. Such arrays may range from a single or double linear electrode to complex multi-electrode arrays. Linear electrodes may be formed from a suitable biocompatible conductive material such as platinum iridium, formed into a linear structure such as a wire, a helical coil or a strip of said material and disposed inside a porous membrane.

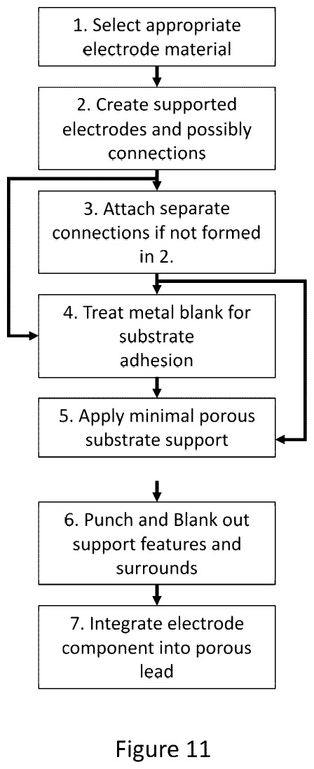

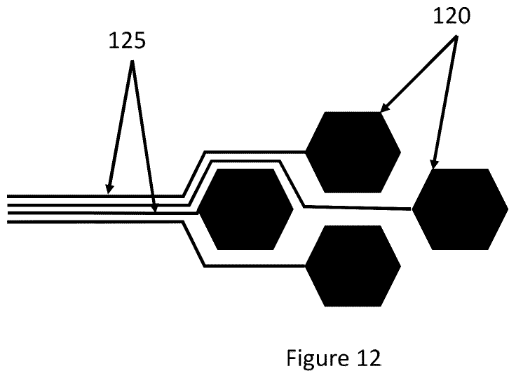

According to another aspect of this invention, arbitrarily complex electrode structures may be formed using the method described in FIG. 11. For the purpose of illustration, a possible complex electrode structure is shown in FIG. 12. This structure has both electrode elements, 120) and connecting elements, 125 which are used to connect the electrode elements, 120, to a stimulation or measurement device.

Once a desired electrode structure has been designed, the method begins with the selection of a suitable electrode material. This choice will be conditioned by the need for biocompatibility, conductivity, safe charge density for the material and the method of forming to be used. There are several possible materials that could be used including, but not limited to, platinum, platinum iridium alloys, tantalum, and stainless steel.

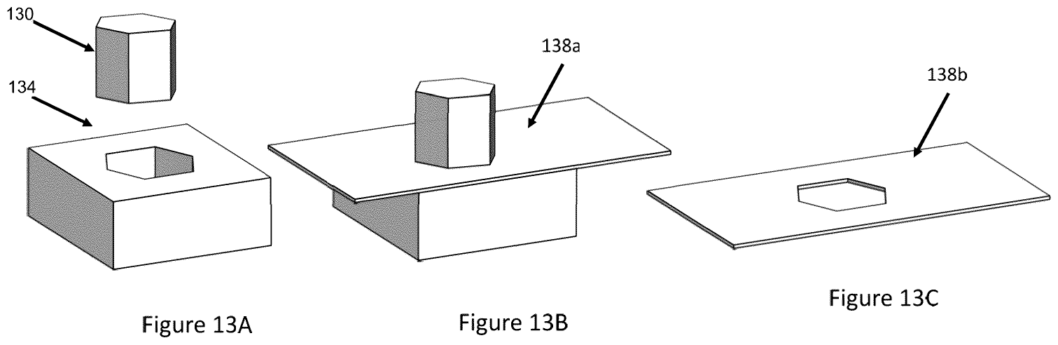

Having selected the appropriate material a “supported electrode” structure is created. For the purpose of illustration a method of manufacture using sheet metal press working tools is described. This method uses one or more forming tools of the type shown in FIGS. 13A-13C. This tool may comprise a punch, 130 and a matching die, 134. A sheet of metal, 138a, is placed on the top face of the die, 134 and the punch, 130, which is aligned to the matching cavity in the die, 134, is pressed down into the die cavity, cutting a hole in the sheet of metal in the shape of the punch, 138b. Those skilled in the art will know this operation to be a cutting operation called “piercing”. Other possible cutting operations using this forming method include blanking, lancing, perforating, trimming, shaving, parting off and broaching. Press working tools may also be used for forming (drawing or bending) and coining (compressing, squeezing or forging) operations. Those skilled in the art of forming complex shapes in sheet materials will understand other methods may also be used in the process. These methods include Electro-Discharge Machining (EDM), laser cutting, milling, chemical etching, and powder metal molding. These and all other such methods for forming complex shapes are covered by this disclosure.

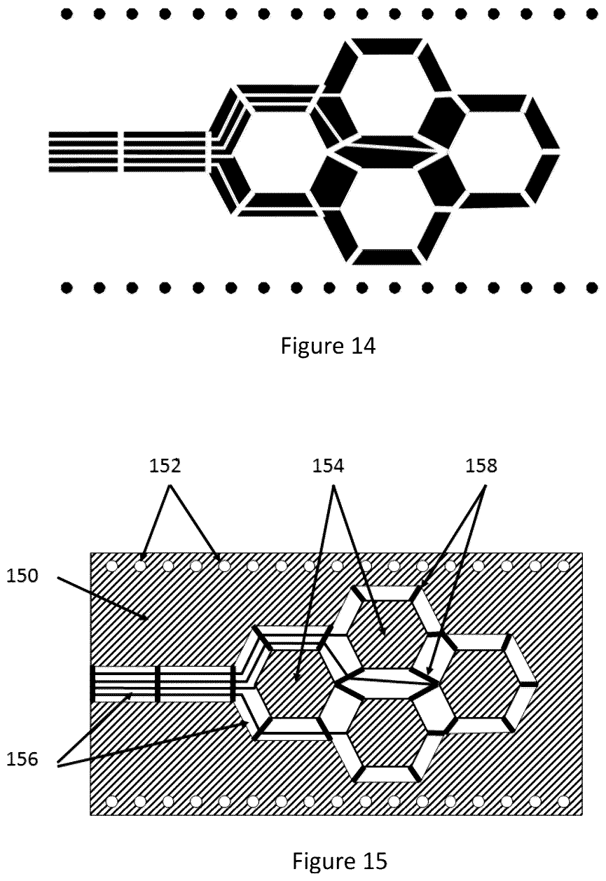

Step 2 in the process described in FIG. 11 is to form what is called the “supported electrode structure”. Using sheet metal press working tools, a piercing punch and die set in the form shown in FIG. 14 is used to form the “supported electrode structure” shown in FIG. 15. Key features of this structure are: a support structure such as an electrode material strip (150); pilot holes (152) created for alignment in future forming operations; the electrode elements (154); possible conducting elements (156) and supporting elements (158) which are used to support the electrode elements (which maintain a desired spacing between adjacent electrode elements) (154) and conducting elements (156) in place until a later stage in the process.

In the event that only the electrode elements (154) are formed in the first forming step, this second step may be followed by a third step that attaches conducting elements to the electrode elements by any of the well-known fixing methods such as welding, brazing and crimping.

Once the supported electrode structure is formed an optional fourth step may be taken to prepare the surface of the supported electrode structure for embedding in a minimal porous substrate (step 4, FIG. 11). Since this surface preparation is done to ensure good adhesion between the minimal porous substrate and the supported electrode structure, the exact nature of the preparation will depend on the materials used for the porous substrate and the electrode structure. Possible treatments include, but are not limited to, one or more of the following coating with a bonding agent, texturing by mechanical means such as bead-blasting, chemical etching, treatment by plasma, for example, an highly dissociated oxygen plasma.

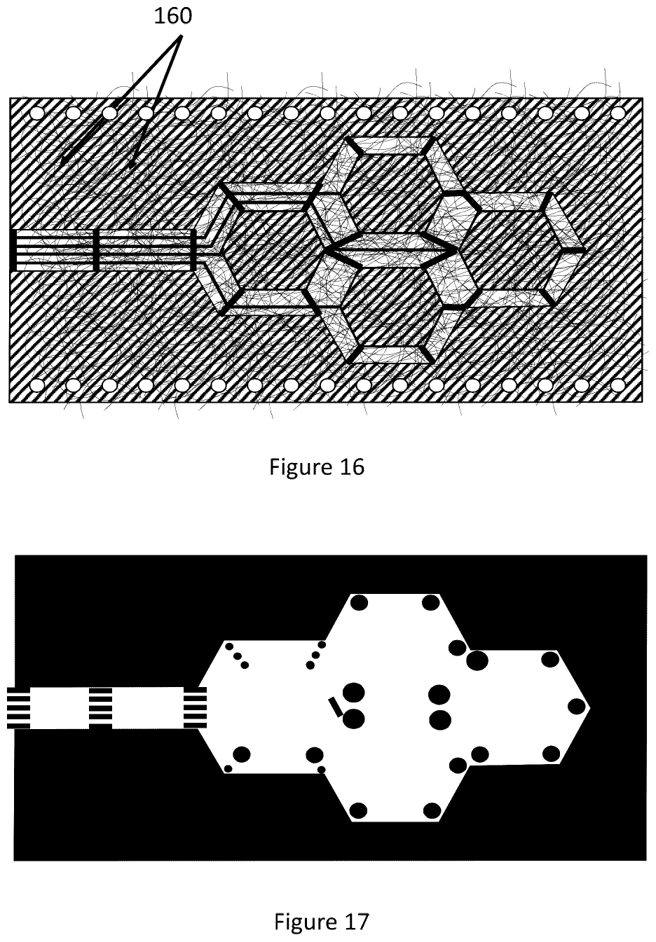

Once any necessary surface treatment has been completed the supported electrode structure is embedded in the appropriate porous substrate (FIG. 11 step 5). The nature of the porous substrate used in this kind of stimulation lead is described in International Patent Cooperation Treaty application PCT/US16/27570. There are various methods for creating this porous substrate, but the preferred embodiment for this invention is the method known as electrospinning. FIG. 16 is a representation of such an embedded supported electrode structure as might result from this step 5. The porous substrate (160) will surround and enmesh or embed the supported electrode structure and will, ideally, adhere to the supported electrode structure as well as filling the voids that were punched in the supported electrode structure in step 2.

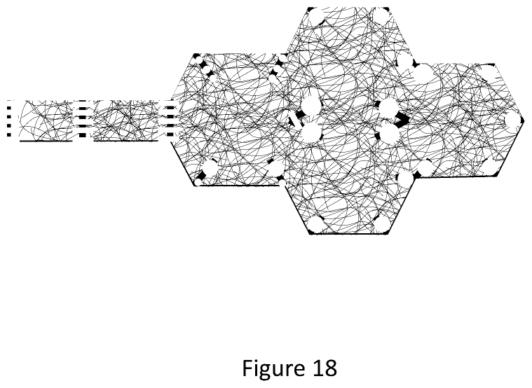

The next step in this process (FIG. 11 step 6) is to remove the supporting elements and surrounding material from the embedded supported electrode while leaving as much of the embedding porous substrate as possible. For illustrative purposes a sheet metal press working tool approach is again used. In the example presented and piercing and blanking press tool in the form shown in FIG. 17 is used to remove the supporting elements and surrounding material from the embedded supported electrode. The resulting structure, represented in FIG. 18, has each electrically isolated electrode and associated conducting element formed and held in place by the embedding porous substrate. Those skilled in the art will understand other methods may also be used for this process. These methods include at least laser cutting and milling. These and all other such methods are covered by this disclosure.

This assembly can then be integrated into a porous lead using methods described in International Patent Cooperation Treaty application PCT/US16/27570 (FIG. 11 step 7).

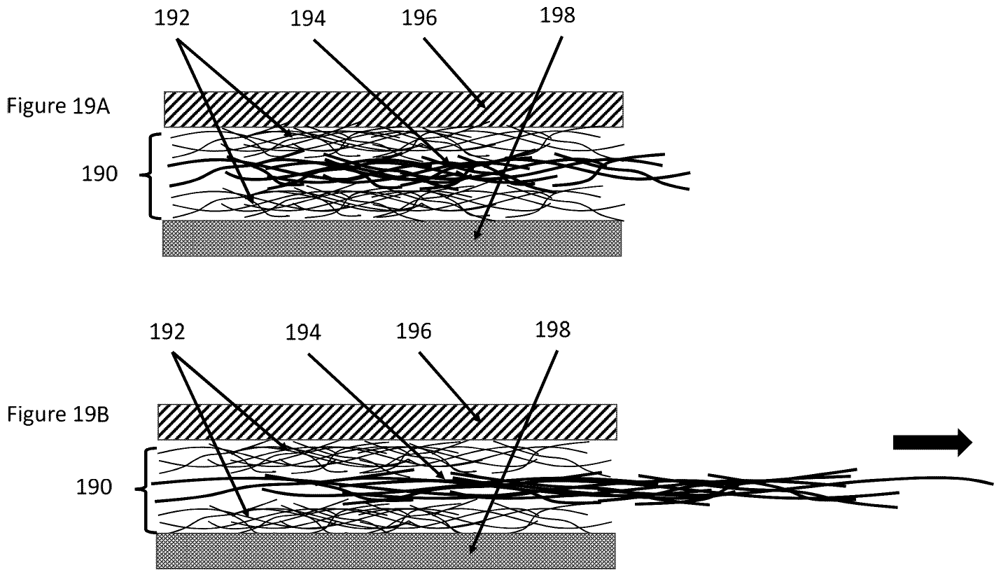

An important characteristic of the ribbon stimulator device based on the porous lead technology is that the biomimetic structure of the substrate can promote cell adhesion to the device. This adhesion has the benefit of stabilizing or anchoring the device where is implanted. This benefit may, however, also make it more difficult to remove the device should explantation be required. According to another aspect of this invention, in some embodiments the porous membrane may be fabricated in a way to assist the removal of a ribbon stimulator device from the body. One method for doing this can be understood with reference to FIG. 19. When it is implanted in tissue, the porous substrate (190) forms the interface between the device (196) and the tissue (198). In the case where the porous substrate is formed with a non-woven fabric, it will be understood that this fabric is made up of many individual fibers, randomly oriented and bound by adhesion to other fibers where they cross. It will be understood the characteristics of the non-woven fabric used as the porous substrate will be strongly affected by the properties of the material from which the fibers are formed and the quality of the bonds formed between the overlapping fibers. For example, a substrate made from Polyethylene Terephthalate with strong bonds will be quite inelastic, while a similar substrate formed from Polyurethane will be quite elastic. Variations between these two extremes can be formed According to this aspect of the invention the porous substrate (190) can be fabricated in a layered structure of different materials such that the interface between the layers can be readily broken. As an illustrative example the porous substrate 190 can be fabricated with a relatively elastic layer (194) sandwiched between two relatively inelastic layers (192). The materials and fabrication process are chosen to ensure relatively weak bonds between the two different layers. In normal use (FIG. 19A) the porous substrate has sufficient integrity to encase and secure the ribbon stimulator device. If the device needs to be removed from the tissue the relatively elastic layer (194) is stretched (FIG. 19B) in the plane of the substrate. Because of the different elasticity between the two layers 192 and 194, this stretching will cause the bonds between the two layers to be substantially broken, and the device can then be removed from the tissue, leaving only an inert “sacrificial” outer layer of the porous substrate which has adhered to the tissue to remain in the tissue. Those skilled in the art will understand that this principle requires one or more interfaces between elastic and inelastic layers to operate and that there are various configurations of this principle that can be imagined.

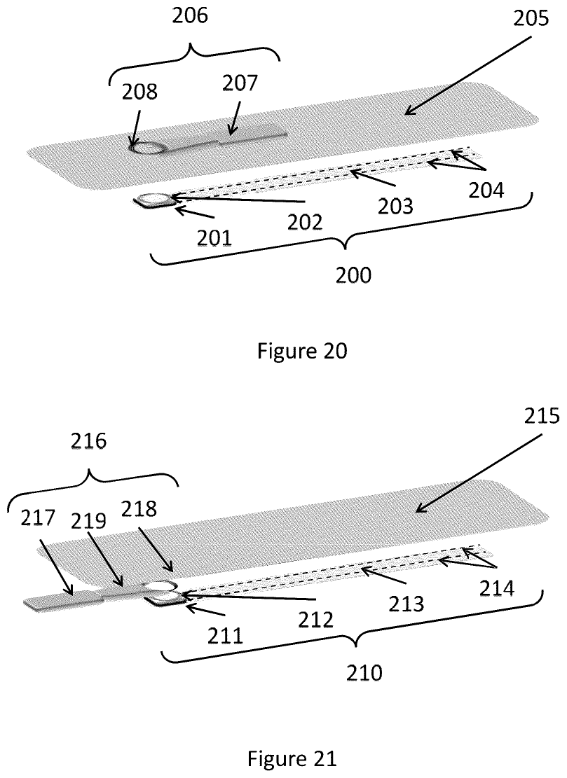

Another important characteristic of a ribbon stimulator device based on porous lead technology are that such devices can be thin and compliant. This means the ribbon stimulator can be reliably implanted in regions of the body which are unsuitable for other neurostimulation leads, such as areas which have little tissue cover (such as the scalp) and areas that suffer high mobility (e.g. close to joints). In a common embodiment of the ribbon stimulator this capacity to be located in otherwise difficult areas can be enhanced by configuring the device as an “RF powered” stimulator. Such a configuration is shown in FIG. 20. The ribbon stimulator (200) is located beneath the skin (205) and an external power source (206) is placed proximal to the ribbon stimulator (200) but external to the skin. The external power source comprises at least an electronics enclosure (207) which has at least a power source and control circuitry to supply a suitable signal to the transmitter coil (208). The transmitter coil (208) is placed proximal to the receiver coil (201) which forms part of the implanted ribbon stimulator (200). When the two coils 201 and 208 are suitable located power can be transmitted between them by coupling of either or both of the electric and magnetic fields generated by the external power source (206).

While the RF configuration allows for ribbon stimulators to be placed in otherwise difficult locations, it does require the patient to wear an external power source which can be inconvenient. According to another aspect of this invention it is possible, in some cases, it may be both possible and desirable to be able to power an RF powered ribbon stimulator with an implanted power source. In this case (FIG. 21) a power source (216) is constructed from biocompatible materials and configured to have at least an electronics enclosure (217) which has at least a power source and control circuitry to a suitable signal to a transmitter coil (218). The transmitter coil (218) is connected to the electronics enclosure (217) by a flexible connection (219) such that the electronics enclosure can be placed.

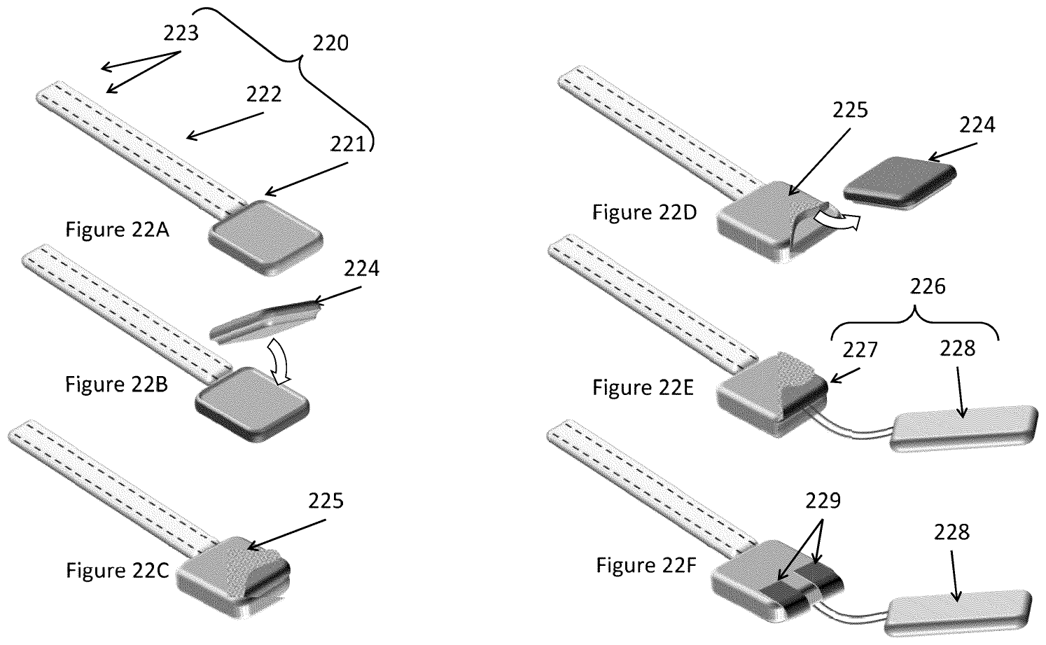

According to another aspect of this invention the ribbon stimulator can be constructed to facilitate the conversion of a ribbon stimulator which uses an external RF power supply to one which uses an implantable power source. With reference to FIGS. 22A-22F, a typical ribbon stimulator (220) comprises an electronics package (221) and electrode structures (223) embedded in the porous substrate (222). The electronics package (221) is constructed with features which facilitate the alignment of another structure with this package (FIG. 22A). In FIG. 22B this feature is shown to be a lip running around the edge of the top of the electronic package (221). For a device that will be configured to be powered by an external RF power supply a dummy alignment component (224) is made from suitable biocompatible material which will not interfere with RF power transfer (FIG. 22B). The dummy alignment component (224) has a matching alignment feature such that the dummy alignment component fits snugly into the alignment feature of the electronics package (221). The combined electronics package (221) and dummy alignment component (224) and then covered by the same porous substrate (225). In FIG. 22C this cover is shown partially cut away to show the internal components (the electronics package (221) and dummy alignment component (224)). This assembly can then be implanted and powered by an external RF power source.

To convert the RF powered ribbon simulator to one with an implanted power source a surgeon exposes the implanted electronics package (221) and dummy alignment component which are covered with the porous membrane (225) cuts an opening in the porous membrane (225) and removes the dummy alignment component (224) (see FIG. 22D). The implanted power source (226) is then introduced. The implanted power source (226) comprises at least a power and electronics assembly (228), which contains the power source to be implanted and such control electronics as are necessary for its proper functioning, and a power coupling assembly (227). The power coupling assembly (227) contains a coupling component such as a coil or antenna to transfer power to the electronics package (221) and is fabricated with similar alignment features as the dummy alignment component (224) so that it will fit snugly into the alignment feature of the electronics package (221). The power coupling assembly is connected to the electronics assembly (228) by suitable electrical cables which are of appropriate length and construction for the placement of the implanted power source (226) as required by the application. This power coupling assembly (227) is placed inside the porous membrane in the void left when the dummy alignment component (224) is removed, and arranged to fit snugly into the alignment feature of the electronics package (221), see FIG. 22E. The opening in the porous membrane cover is then closed using a suitable closure device (229) and the electronics assembly implanted in a suitable location proximal to the ribbon stimulator (220), and the wound is closed.

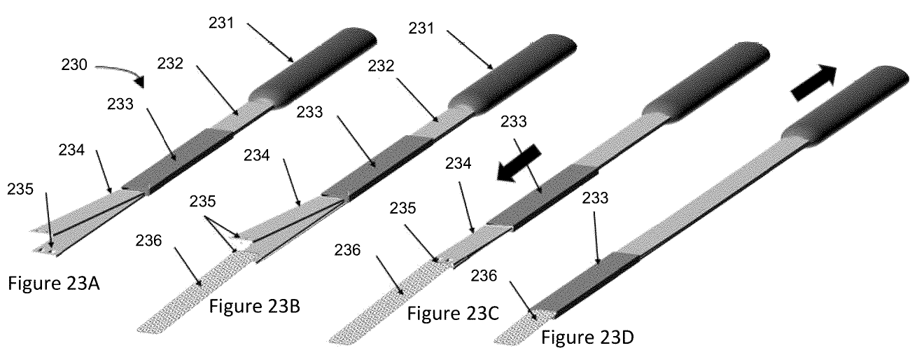

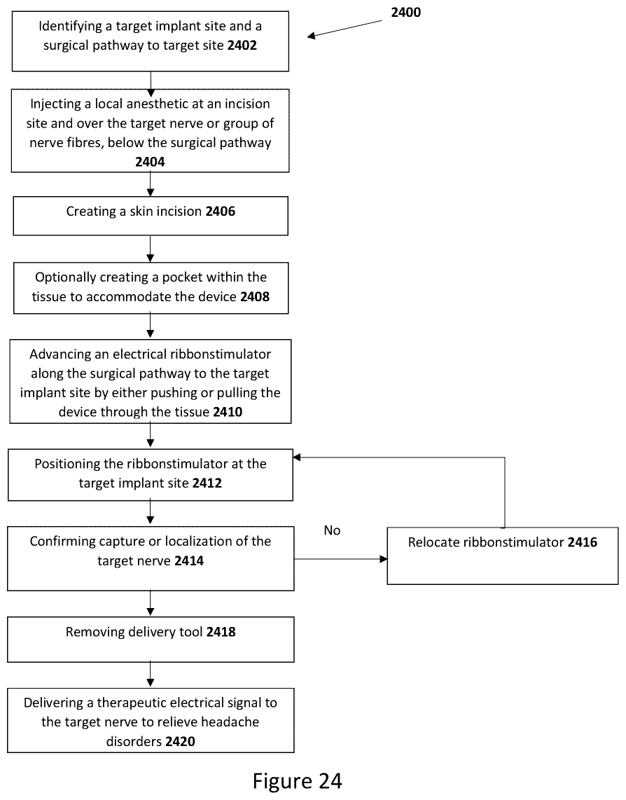

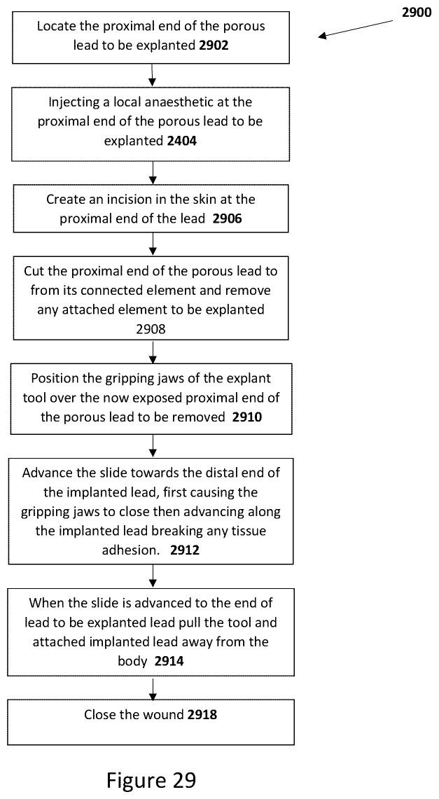

In the event of adverse events, it may be necessary to remove an implanted ribbon stimulator from a patient's body. This process is generally referred to as explanation. Because of the biomimetic structure of the porous substrate that encloses most or all of the ribbon stimulator, a device that has been implanted for any length of time will have adhered to the surrounding tissue and will resist being simply pulled out. According to another aspect of this invention, a special “explant tool” can be used to solve this problem. With reference to FIG. 23A, and FIG. 29 the explant tool 220 consists of at least: a handle, 221, suitable for a surgeon to easily grip and hold; a shank, 222, which has a cross sectional form which is generally the same or similar to the cross-sectional area of the ribbon stimulator to be explanted and of appropriate length; a slide, 223 which is configured to be a snug fit over the shank, 222, and which has a rounded or blunt edge on the end facing towards the gripping jaws, 224, and which can be slid past the end of the gripping jaws, 224, for at least the length of the ribbon stimulator to be explanted; gripping jaws, 224, which are disposed to be held open with a light to moderate spring force; and one or more gripping features, 225 suitable for engaging with the ribbon stimulator material without tearing.

In use, with reference to FIG. 23B an incision is made at or near to the end of the ribbon stimulator and the end is exposed. The slide, 223, of the explant tool, 220, is withdrawn from the jaws, 224, so they are in the open position and the exposed end of the ribbon stimulator, 226, is positioned between gripping features, 225, of the jaws 224. With reference to FIG. 23C, the slide, 223, is then advanced along the shank, 222, of the tool, causing the jaws, 224, to close the gripping features to engage with the ribbon stimulator, 226, This has the effect of aligning and engaging and aligning the end of the ribbon stimulator, 226, to the shank, 222, of the explant tool, 220. With the explant tool, 220, so aligned the slide, 223, is further advanced into the tissue, forcing the rounded or blunt edge along the face of the ribbon stimulator and breaking the adhesion between the ribbon stimulator and the surrounding tissue. With reference to FIG. 23D, once the slide, 223, is fully extended, the adhesion between the ribbon stimulator and the surrounding tissue has been fully broken. While the slide, 223, is in the fully extended position the explant tool, 220, is withdrawn taking with it ribbon stimulator. The explant wound can then be sutured closed and dressed.

Embodiments as disclosed herein are directed to delivering PNS for pain of peripheral origin and other disorders, and in particular improving headache disorders. Headache disorders can refer to but are not limited to migraine, chronic migraine, episodic migraine, cluster headache, chronic cluster headache, episodic cluster headache.

The International Headache Society defines chronic migraine as more than fifteen headache days per month over a three-month period of which more than eight are migrainous, in the absence of medication over use. Episodic migraine is the other migraine sub-type, which is defined as less than 15 headache days per month. Symptoms of migraine include moderate to severe headaches, headache on one side of the head only, pulsating headache pain, headaches aggravated by routine physical activity, and headaches that cause nausea, vomiting or both.

As stated above, embodiments as disclosed herein differ from other methods of treating headache disorders. Current ONS involve placing an SCS electrode above the muscle over the occipital nerve at the back of the head. Because of the stiffness and large diameter of the current SCS electrodes they are not suitable to placement high on the back of the head. This placement is further from the target nerve and has a high probability of recruiting muscle and painful sensory fibers. In addition, these electrodes are connected to a distal implantable pulse generator (IPG) implanted in the patients torso. This requires tunneling of connecting wires between the electrodes and the IPG. Adverse event such as lead migration, lead infection, lead breakage etc. are common. Traditional ONS requires invasive surgery such as tunneling and creating a large pocket for the IPG. Embodiments of devices and methods as described herein provide a minimally invasive yet targeted and efficacious form of therapy for improving headache disorders.

The implantation site for the proposed product is illustrated in FIG. 3. The ribbon stimulator is an all “on-head” device that is implanted subcutaneously below the occipital ridge in a procedure that is far less invasive than the current practice. By making the lead structures thin and flexible and having no leads traversing the neck this design addresses all the significant root causes of ADEs. The external transmitter device provides power to the implanted device by magnetic induction. This device, shown in green in the figure, is configured like a “sports headphone” and is supported on the ears. Other transmitter configurations will be considered. Powering the device with an external transmitter allows the implanted device to be much smaller, likely reducing any device-related discomfort.

Each of the disclosed aspects and embodiments of the present disclosure may be considered individually or in combination with other aspects, embodiments, and variations of the disclosure. Unless otherwise specified, none of the steps of the methods of the present disclosure are confined to any particular order of performance.

The present invention involves a method and a therapeutic or stimulation system having a surgically implanted device in communication with a predetermined site, selected from the group consisting of C2 dermatome area, C3 dermatome area, cervical nerve roots (e.g., C1, C2, C3, C4, C5, C6, C7 and C8) and cranial nerves (e.g., olfactory nerve, optic, nerve, oculomotor nerve, trochlear nerve, trigeminal nerve, abducent nerve, facial nerve, vestibulocochlear nerve, glossopharyngeal nerve, vagal nerve, accessory nerve, and hypoglossal nerve). The device is operated to stimulate the predetermined site thereby treating neurological disorder. The device can include a probe, for example, a porous electrode and/or a porous electrode assembly (e.g., electrical stimulation lead). The proximal end of the probe is coupled to a pulse generating source, (e.g., an electrical signal source), which, in turn, is operated to stimulate the predetermined treatment site, in certain embodiments, the probe and the pulse generating source are contained within the same unit, for example the Bion® stimulation system manufactured by Advanced Bionics Corporation, in addition, the system may include a means to program the pulse generating source, for example the means may include a device that allows for external programming, such as a hand-held device. An electrical stimulation system having one or more stimulation porous electrodes is implanted subcutaneously such that one or more of the stimulation porous electrodes are in communication with the C2 and/or C3 dermatome area. More particularly, the stimulation porous electrodes are in communication with the C2 and/or C3 dermatome area or any of the branches or terminal branches of the C2 and/or C3 dermatome area located in the C2 and/or C3 dermatome area or any cervical nerve roots located in the C2 and/or C3 dermatome area and/or any cranial nerves that may be proximate or within or adjacent to the C2 and/or C3 dermatome area. The one or more stimulation porous electrodes deliver electrical stimulation pulses to the neuronal tissue of the C2 and/or C3 dermatome area, which thereby permanently or temporarily eliminates, reduces, or otherwise treats the one or more neurological disorders. This may in turn significantly increase the person's quality of life. Although headache disorder may be treated in combination with the one or more conditions treated according to the present invention, in such a case the one or more conditions treated will include one or more conditions in addition to headache disorder.

In certain embodiments, electrical stimulation of the predetermined site for example, C2 dermatome area. C3 dermatome area, cervical nerve roots and/or cranial nerves, may be provided to effectively treat pain. For example, in certain embodiments, electrical stimulation of the predetermined site may be provided to effectively treat fibromyalgia and/or chronic fatigue syndrome or other diffuse pain in any one or more regions of the body. As another example, in certain embodiments, electrical stimulation of the predetermined site maybe delivered to treat localized, diffuse, or other pain in any one or more regions of the body below the head, such as pain in the neck, shoulders, upper extremities, torso, abdomen, hips, and lower extremities. As another example, in certain embodiments, electrical stimulation of the predetermined site may be delivered to treat Reflex Sympathetic Dystrophy (RSD) pain. As another example, in certain embodiments, electrical stimulation of the predetermined site may decrease the person's overall sensitivity to pain or increase the person's overall pain threshold, in certain cases significantly, such that the person experiences “total body” pain relief or other generalized pain relief throughout the body. For example, a person with a relatively low overall pain threshold may experience an elevation of the pain threshold from a relatively hyperalgesic state to a relatively normalized state, with concomitant pain relief throughout the body. Other example pain-related applications of electrical stimulation of the predetermined site in certain embodiments include: (1) treating post-operative pain associated with major surgery, perhaps using a temporary as opposed to a permanent stimulation lead (e.g., to augment or replace opioid analgesia); (2) treating focal pain (e.g., possibly in combination with electrical stimulation of the spinal cord or peripheral structures such as the periostium around the knee or hip); (3) treating pain in elderly patients with severe degenerative spinal or joint conditions (e.g., with additional improvements in sleep, cognition, and mood); and (4) treating trigeminal neuralgia. Yet further, the stimulation system of the present invention may result in pain relief in areas of the head not innervated by the C2 dermatome area and/or C3 dermatome area (such as outside the C2 dermatome area), for example, but not limited to pain in the face, ears, and mouth. These areas are innervated by the trigeminal nerve and other cranial nerves and those of the cervical plexus.

In certain embodiments, possibly in combination with one or more of the benefits described above, electrical stimulation of the C2 and/or C3 dermatome area and/or cervical nerve roots and/or cranial nerves may be provided to effectively treat impaired motor functioning. For example, in certain embodiments, electrical stimulation of the predetermined site may be provided to effectively treat lack of coordination in the upper or lower extremities (e.g., gait problems). As another example, in certain embodiments, electrical stimulation of the predetermined site may be provided to effectively treat motor disorders such as tremor (e.g., reducing the coarseness of tremor, and Parkinson's disease), dystonia (e.g., reducing the frequency and severity of torticollis or other forms of dystonia), restless leg syndrome and seizure.

In certain embodiments, possibly in combination with one or more of the benefits described above, electrical stimulation of the predetermined site may be provided to effectively treat other neurological disorders for example, but not limited to Developmental Disabilities [e.g., Cerebral Palsy, Mental Retardation, Attention Deficit Disorder (ADD), Pervasive Developmental Disorders and Autistic Spectrum Disorders (e.g., autism and Asperger's disorder), Learning Disabilities (e.g., dyslexa disorders of motor functions (e.g., dysgraphia, dyspraxia, clumsiness), and nonverbal learning disabilities (e.g., dyscalculia, visuospatial dysfunction, socioemotional disabilities, and ADHD)]; Demyleinating Diseases [e.g., Multiple Sclerosis]; delirium and dementia [e.g., vascular dementia, dementia due to Parkinson's disease, dementia due to HIV disease, dementia due to Huntington's disease, and dementia due to Creutzfeld-Jakob disease; Alzheimer's dementia, multi-infarct dementia, stroke]; affective disorder [e.g., depression, mania, mood disorder, major depressive disorder, bipolar]; movement disorders [e.g, restless leg syndrome, Dyskinesia (e.g., tremor, dystonia, chorea and ballism, tic syndromes (e.g., Tourette's Syndrome), myoclonus, drug-induced movement disorders, Wilson's Disease, Paroxysmal Dyskinesias, Stiff Man Syndrome) and Akinetic-Ridgid Syndromes and Parkinsonism]; ataxic disorders [e.g., disturbances of gait]; substance abuse related disorders [e.g., alcohol use disorders, amphetamine use disorders, cannabis use disorders, caffeine induced disorders, cocaine use disorders, inhalant use disorders, opioid use disorders, hallucinogen disorders, sedative, hypnotic, or anxiolytic use disorders, and polysubstance use disorders]; sexual dysfunctions [e.g., sexual arousal disorder, male erectile disorder, female dyspareunia, male hypoactive disorder, and female hypoactive disorder]; eating disorders [e.g., overeating disorder, bulimia nervosa, and anorexia nervosa]; obesity, anxiety and obsessive compulsive disorder syndromes [e.g., anxiety, panic attacks, post-traumatic stress disorder, agoraphobia, obsessive and compulsive behavior]; impulse control disorders [e.g., pathological gambling, intermittent explosive disorder, kleptomania, and pyromania]; personality disorders (e.g., schizoid personality disorder, paranoid personality disorder, schizotypal personality disorder, borderline personality disorder, narcissistic personality disorder, histrionic personality disorder, obsessive compulsive personality disorder, avoidant personality disorder, dependent personality disorder, and anti-social personality disorder); and other psychiatric disorders [e.g., schizophrenia subtypes, schizoaffective disorder, schizophrenia undifferentiated, delusional disorder, cyclothymic disorder, somatoform disorder, hypochondriasis, dissociative disorder, and depersonalization disorder]; and Chiari I malformation.

In certain embodiments, the stimulation system of the present invention can be used to treat obesity and obesity related conditions and/or gastric motility conditions. The gastrointestinal disorders or conditions contemplated by the present invention include gastrointestinal altered motility, sensitivity and secretion disorders in which one or more of the symptoms and conditions affect the gastrointestinal tract from the mouth to the anus. Gastrointestinal disorders include, but are not limited to, heartburn, bloating, postoperative ileus, abdominal pain and discomfort, early satiety, epigastric pain, nausea, vomiting, burbulence, regurgitation, intestinal pseudoobstruction, anal incontinence, gastroesophageal reflux disease, irritable bowel syndrome, ulcerative colitis, Crohn's disease, menstrual cramps, pancreatitis, spastic and interstitial cystitis and ulcers and the visceral pain associated therewith. One with skill in the art is aware that any functional gastrointestinal disorder, including but not limited to those associated with gastric motility, is appropriate for treatment with the method and systems of the present invention. Eating disorders and conditions can include, but are not limited to obesity, anorexia nervosa, and bulimia nervosa. For example, it is contemplated that the method of the present invention may be used to treat a patient for obesity, binge eating, or compulsive overeating.

Yet further other conditions that can be treated include immune-diseases. Immune-mediated diseases include, for example, but not limited to, arthritis (e.g., rheumatoid arthritis and psoriatic arthritis), inflammatory bowel diseases (e.g., ulcerative colitis and Crohn's disease), endocrinopathies (e.g., type 1 diabetes and Graves disease), neurodegenerative diseases (e.g., multiple sclerosis, autistic spectrum disorder, Alzheimer's disease, Guillain-Barre syndrome, obsessive-compulsive disorder, optic neuritis, retinal degeneration, amyotrophic lateral sclerosis (ALS), Parkinson's disease, Huntington's Disease, Guillain-Barre syndrome, myasthenia gravis, and chronic idiopathic demyelinating disease (CID)), vascular diseases (e.g., autoimmune hearing loss, systemic vasculitis, and atherosclerosis), and skin diseases (e.g., dermatomyositis, systemic lupus erthematosus, discoid lupus erthematosus, scleroderma, and vasculitics). More specifically, the immune-mediated disorders that can be treated by the present invention include, but are not limited to rheumatoid arthritis, eryematos, Sojourn's syndrome, allergic asthma, atopic skin disease, chronic fatigue syndrome, allergies, and Chron's disease.

In certain aspects of the present invention, the stimulation system can be used to treat neuroendocrine disorders, such as disorders associated with the crosstalk that occurs between the endocrine system and the nervous system. More particularly, the stimulation system of the present invention can be used to treat disorders associated with the hypothalamic-pituitary-adrenal (HPA) and-gonadal (HPG) axes, as well as disorders associated with the autonomic nervous system. Diseases associated with the HPA axis can include, but are not limited to pituitary tumors, Cushing syndrome, adrenal insufficiency, ACTH resistance, Congenital Adrenal Hyperplasia (CAH), adrenocortical tumors, glucocorticoid resistance/hypersensitivity, and mineralocorticoid resistance. Diseases of the HPG axis can include, but are not limited to hypothalamic hypogonadism, disturbances of the menstrual cycle, ovarian and testicular gonadotropin resistance, endometriosis, and infertility. Disease associated with the autonomic nervous system can include, but are not limited to pheochromocytoma and catecholamine deficiency. Still further, developmental/psychiatric, metabolic and immune disorders related to the functions of the HPA and HPG axes and the autonomic system can include, but are not limited to premature adrenarche, eating disorders—including anorexia and bulimia nervosa and adolescent obesity—adolescent conduct disorder, dysthymia and depression, childhood asthma and rheumatoid arthritis, the premenstrual tension syndrome, and postpartum and climacteric depression and autoimmunity.

Still further, the stimulation system of the present invention can be used to provide stimulation to the predetermined sited to enhance or improve cardiac function, for example, hemodynamics, electrical activity, myocontractility, perfusion of the heart muscle, as well as enhance cardiac performance or efficiency, such as balance between supply and demand. Thus, the present invention can be used as a prophylactic system to enhance or improve cardiac function. The present invention may also be used to treat cardiovascular disorders which include but are not limited to diseases and/or disorders of the pericardium (i.e., pericardium), heart valves (i.e., incompetent valves, stenosed valves, Rheumatic heart disease, mitral valve prolapse, aortic regurgitation), myocardium (coronary artery disease, myocardial infarction, heart failure, ischemic heart disease, angina) blood vessels (i.e., arteriosclerosis, aneurysm) or veins (i.e., varicose veins, hemorrhoids). Other symptoms that can be related cardiovascular diseases include cholesterol and/or blood pressure. Thus, the present invention can be used to decrease cholesterol levels and/or to regulate blood pressure, for example decrease blood pressure in a subject suffering from high blood pressure.

Yet further, the stimulation system of the present invention can be use to provide stimulation to the predetermined site to modulate blood glucose. Blood glucose can be used as an indicator of diabetes mellitus. Thus, it is envisioned that the present invention may be used to treat diabetes mellitus.

In certain embodiments, electrical stimulation of the predetermined area may effectively treat other conditions including intractable nausea, chronic fatigue, sleep disorders, sleep apnea, visceral disorders, such as irritable bowel or areas of the body supplied and controlled mainly by the autonomic nervous system.

In certain embodiments, electrical stimulation of the predetermined area may effectively treat one or more neurological disorder associated with traumatic brain injury (TBI). Physiological conditions associated with TBI that may be treated effectively through electrical stimulation of the C2 and/or C3 dermatome area include, for example, intractable localized, diffuse, or other pain in the head, neck, shoulders, upper extremities, or low back, fibromyalgia chronic syndrome fatigue, or other diffuse pain in one or more regions of the body, or other pain symptoms. Instead or in addition to such physiological conditions, psychological and other conditions associated with TBI that may be treated effectively through electrical stimulation of the C2 and/or C3 dermatome area include, for example, intractable nausea {e.g., from gastroparesis), sleep disorders, sleep apnea, immune-mediated disorders, inflammatory disorders, cardiovascular disorders, improve cardiac function and/or performance, chronic fatigue, behavioral modifications {e.g., lassitude, reduced motivation, depression, emotional distress, irritability, aggression, anxiety, erratic mood swings, personality changes, and loss of enjoyment), sexual dysfunction, and other conditions. Instead or in addition to physiological, psychological, and other conditions such as those described above, conditions associated with TBI that may be treated effectively through electrical stimulation of the C2 and/or C3 dermatome area include decreased cognitive functioning in the form of, for example, impaired memory {e.g., short-term memory, visual memory, and auditory memory), reduced attention and concentration, and reduced information processing capacity {e.g., learning capacity, ability to process complex information, ability to operate simultaneously on different information, ability to rapidly shift attention, ability to plan and sequence, visuomotor capability, auditory language comprehension, and verbal fluency).

In certain embodiments, qEEG analysis may be performed before and after electrical stimulation to determine whether and the extent to which changes in the brain and associated improvements in cognitive functioning effected during electrical stimulation persist after electrical stimulation ceases and for how long. In certain embodiments, the electrical stimulation may modify the brain such that the qEEG is shifted towards normal, as reflected in a normative reference database for example, or otherwise influenced in a positive manner. In other embodiments, it is envisioned that the qEEG and porous electrode stimulating device may be an all in one type system.

In certain embodiments, one or more appropriate neuropsychological measures may also be used to assess the short term and long term impact of treatment. Observations of improved cognitive function in persons treated according to the present invention suggest that electrical stimulation of the C2 and/or C3 dermatome area may provide a level of cortical stimulation sufficient to maintain cognitive gains even after electrical stimulation ceases. The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

In certain embodiments the stimulation system having a surgically implanted device in communication with a predetermined site, selected from the group consisting of T1 dermatome area, T2 dermatome area, thoracic nerve roots (e.g., T1, T2, T3, T4, T5, T6, T7, T8, T9, T10, T11 and T12) and thoracic nerves (e.g., the anterior divisions of the thoracic nerves (rami anteriores; ventral divisions), thoracic intercostal nerves, thoracicoabdominal intercostal nerves, anterior cutaneous branches of the abdomensciatic nerve).

In certain embodiments, electrical stimulation of the predetermined site for example, T8 dermatome area, T9) dermatome area, thoracic nerve roots and/or thoracic nerves, may be provided to effectively treat pain. For example, in certain embodiments, electrical stimulation of the predetermined site may be provided to effectively treat back pain. As another example, in certain embodiments, electrical stimulation of the predetermined site maybe delivered to treat localized, diffuse, or other pain in any one or more regions of the back and legs, such as pain as the result of FBSS, RSD, radiculopathies, degenerative disks, and back injuries. As another example, in certain embodiments, electrical stimulation of the predetermined site may be delivered to treat Reflex Sympathetic Dystrophy (RSD) pain. As another example, in certain embodiments, electrical stimulation of the predetermined site may decrease the person's overall sensitivity to pain or increase the person's overall pain threshold, in certain cases significantly, such that the person experiences pain relief or other generalized pain relief throughout the lower body. For example, a person with a relatively low overall pain threshold may experience an elevation of the pain threshold from a relatively hyperalgesic state to a relatively normalized state, with concomitant pain relief throughout the body. Other example pain-related applications of electrical stimulation of the predetermined site in certain embodiments include: (1) treating post-operative pain associated with major surgery, perhaps using a temporary as opposed to a permanent stimulation lead (e.g., to augment or replace opioid analgesia); (2) treating focal pain (e.g., possibly in combination with electrical stimulation of the spinal cord or peripheral structures such as the periostium around the knee or hip); (3) treating pain in elderly patients with severe degenerative spinal or joint conditions (e.g., with additional improvements in sleep, cognition, and mood); and (4) treating neuralgia.

In certain embodiments, electrical stimulation of the predetermined site for example, T1 dermatome area, T2 dermatome area, thoracic nerve roots and/or thoracic nerves, may be provided to effectively treat to enhance or improve cardiac function, for example, hemodynamics, electrical activity, myocontractility, perfusion of the heart muscle, as well as enhance cardiac performance or efficiency, such as balance between supply and demand. Thus, the present invention can be used as a prophylactic system to enhance or improve cardiac function. The present invention may also be used to treat cardiovascular disorders which include but are not limited to diseases and/or disorders of the pericardium (i.e., pericardium), heart valves (i.e., incompetent valves, stenosed valves, Rheumatic heart disease, mitral valve prolapse, aortic regurgitation), myocardium (coronary artery disease, myocardial infarction, heart failure, ischemic heart disease, angina) blood vessels (i.e., arteriosclerosis, aneurysm) or veins (i.e., varicose veins, hemorrhoids). Other symptoms that can be related cardiovascular diseases include cholesterol and/or blood pressure. Thus, the present invention can be used to decrease cholesterol levels and/or to regulate blood pressure, for example decrease blood pressure in a subject suffering from high blood pressure.

In certain embodiments, the stimulation system of the present invention can be used to treat gastrointestinal disorders or conditions contemplated by the present invention include gastrointestinal altered motility, sensitivity and secretion disorders in which one or more of the symptoms and conditions affect the gastrointestinal tract from the mouth to the anus. Gastrointestinal disorders include, but are not limited to, heartburn, bloating, postoperative ileus, abdominal pain and discomfort, early satiety, epigastric pain, nausea, vomiting, burbulence, regurgitation, intestinal pseudoobstruction, anal incontinence, gastroesophageal reflux disease, irritable bowel syndrome, ulcerative colitis, Crohn's disease, menstrual cramps, pancreatitis, spastic and interstitial cystitis and ulcers and the visceral pain associated therewith. One with skill in the art is aware that any functional gastrointestinal disorder, including but not limited to those associated with gastric motility, is appropriate for treatment with the method and systems of the present invention. Eating disorders and conditions can include, but are not limited to obesity, anorexia nervosa, and bulimia nervosa. For example, it is contemplated that the method of the present invention may be used to treat a patient for obesity, binge eating, or compulsive overeating.

Yet further, the stimulation system of the present invention can be use to provide stimulation to the predetermined site to modulate blood glucose. Blood glucose can be used as an indicator of diabetes mellitus. Thus, it is envisioned that the present invention may be used to treat diabetes mellitus.

In certain embodiments, electrical stimulation of the predetermined area may effectively treat other conditions including intractable nausea, chronic fatigue, sleep disorders, sleep apnea, visceral disorders, such as irritable bowel or areas of the body supplied and controlled mainly by the autonomic nervous system.

In certain embodiments the stimulation system having a surgically implanted device in communication with a predetermined site, selected from the group consisting of S2 dermatome area, S3 dermatome area, lumbar and sacral nerve roots (e.g., L1, L2, L3, L4, L5, S1, S2, S3, S4, and S5) and lumbar and sacral nerves (e.g., sciatic nerve, femoral nerve, nerve to quadratus femoris and gemellus inferior: L4-S1, nerve to obturator internus and gemellus superior: L5-S2, nerve to piriformis: S1, S2, superior gluteal nerve: L4-S1, inferior gluteal nerve: L5-S2, posterior femoral cutaneous nerve: S1-S3, tibial nerve: L4-S3, common fibular (peroneal): L4-S2).

In certain embodiments, electrical stimulation of the predetermined site for example, S2 dermatome area, S3 dermatome area, sacral nerve roots and/or sacral nerves, may be provided to effectively treat pain. For example, in certain embodiments, electrical stimulation of the predetermined site may be provided to effectively treat leg pain. As another example, in certain embodiments, electrical stimulation of the predetermined site may be delivered to treat localized, diffuse, or other pain in any one or more regions of the lower body below the waist, such as pain in the leg, pelvis, bladder, GI tract, reproductive organs, and foot. As another example, in certain embodiments, electrical stimulation of the predetermined site may be delivered to treat Reflex Sympathetic Dystrophy (RSD) pain. As another example, in certain embodiments, electrical stimulation of the predetermined site may decrease the person's overall sensitivity to pain or increase the person's overall pain threshold, in certain cases significantly, such that the person experiences pain relief or other generalized pain relief throughout the lower body. For example, a person with a relatively low overall pain threshold may experience an elevation of the pain threshold from a relatively hyperalgesic state to a relatively normalized state, with concomitant pain relief throughout the body. Other example pain-related applications of electrical stimulation of the predetermined site in certain embodiments include: (1) treating post-operative pain associated with major surgery, perhaps using a temporary as opposed to a permanent stimulation lead (e.g., to augment or replace opioid analgesia); (2) treating focal pain (e.g., possibly in combination with electrical stimulation of the spinal cord or peripheral structures such as the periostium around the knee or hip): (3) treating pain in elderly patients with severe degenerative spinal or joint conditions (e.g., with additional improvements in sleep, cognition, and mood): and (4) treating neuralgia.

Still further, the stimulation system of the present invention can be used to provide stimulation to the predetermined sited to enhance or improve bladder or bowel function, for example, reduce urinary or fecal incontinence, reduce nocturia, improve bladder capacity, reduce urgency, as well as normalize bladder and bowel function Thus, the present invention can be used as a prophylactic system to enhance or improve bladder and bowel function. The present invention may also be used to treat gynecological disorders which include but are not limited to diseases and/or disorders of the uterus, or fallopian tubes. The present invention may also be used to treat sexual disorders.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A ribbon stimulator for targeting superficial nerves comprising:
an elongate ribbon body configured to be cut at a predetermined length;
one or more electrodes formed on the elongate ribbon body, wherein the elongate ribbon body is configured to position the one or more electrodes at a target implant site; and
electrical circuitry in communication with the one or more electrodes, wherein when the elongate ribbon body is cut, the one or more electrodes remain in communication to the electrical circuitry.
